SEQUENCE LISTING (66 sequences)

SEQ ID NO: 1 - protein, 36 aa, Artificial Sequence
  Generic peptide
  PEPTIDE (1)..(2): Xaa at residue 1 is des-Xaa, Pro, Hyp, Arg, Lys, ornithine, homo-Lys, homoarginine, nor-Lys, N-methyl-Lys, N,N′-dimethyl-Lys, N,N′,N′′-trimethyl-Lys or any synthetic basic amino acid; Xaa at residue 2 is des-Xaa, Ala, Gly, Asp, Glu, Gla, any
  PEPTIDE (2)..(4): synthetic acidic amino acid, Thr, Ser, g-Thr, g-Ser, Trp (D or L), neo-Trp or halo-Trp (D or L) or Xaa may be pyro-Glu if Xaa at residue 1 is des-Xaa; Xaa at residue 4 is Arg, Lys, ornithine, homo-Lys, homoarginine, nor-Lys,
  PEPTIDE (4)..(4): N-methyl-Lys, N,N′-dimethyl-Lys, N,N′, N′′-trimethyl-Lys, any synthetic basic amino acid, Ser, Thr, g-Ser, g-Thr, Ala, an aliphatic amino acids bearing linear or branched saturated hydrocarbon chains such as Leu (D or L), Ile an
  PEPTIDE (4)..(5): and Val or non-natural derivatives of the aliphatic amino acid, H is, Glu, Gln, Gla, Asp, Asn or any synthetic acidic amino acid; X aa at residue 5 is Glu, Gla, Gln, Asp, Asn, any synthetic acidic amino acid, Lys, Arg, ornithine
  PEPTIDE (5)..(5): homo-Lys, homoarginine, nor-Lys, N-methyl-Lys, N,N′-dimethyl-Lys, N,N′,N′′- trimethyl-Lys, any synthetic basic amino acid, Ala, an aliphatic amino acids bearing linear or branched saturated hydrocarbon chains such as Leu (D or L),
  PEPTIDE (5)..(6): Ile and Val or non-natural derivatives of the aliphatic amino acid, Ser, Thr, Pro, Hyp, g-Ser, g-Thr, g-Hyp or any synthetic hydroxylated amino acid; Xaa at residue 6 is Lys, Arg, ornithine, homo-Lys, homoarginine, nor-Lys,
  PEPTIDE (6)..(6): N-methyl-Lys, N,N′-dimethyl-Lys, N,N′, N′′-trimethyl-Lys, any synthetic basic amino acid, Tyr, meta-Tyr, ortho-Tyr, nor-Tyr, mono-halo-Tyr, di-halo-Tyr, O-sulpho-Tyr, O-phospho-Tyr, nitro-Tyr, an aliphatic amino acids bearing linear or
  PEPTIDE (6)..(7): or branched saturated hydrocarbon chains such as Leu (D or L), Ile and Val or non-natural derivatives of the aliphatic amino acid, Glu, Gla, Gln, Asp, Asn, any synthetic acidic amino acid, Pro or Hyp; Xaa at residue 7 is Trp (D or L),
  PEPTIDE (7)..(8): neo-Trp, halo-Trp (D or L), Gly, Tyr, meta-Tyr, ortho-Tyr, nor-Tyr, mono-halo-Tyr, di-halo-Tyr, O-sulpho-Tyr, O-phospho-Tyr, nitro-Tyr, Glu, Gla, Gln, Asp, Asn, any synthetic acidic amino acid; X aa at residue 8 is Glu, Gla, Gln, Asp, Asn,
  PEPTIDE (8)..(8): any synthetic acidic amino acid, Met, norleucine (Nle), Ala, an a liphatic amino acids bearing linear or branched saturated hydrocarbon chains such as Leu (D or L), Ile and Val or non-natural derivatives of the aliphatic amino acid, Tyr,
  PEPTIDE (8)..(8): meta-Tyr, ortho-Tyr, nor-Tyr, mono-halo-Tyr, di-halo-Tyr, O-sulpho-Tyr, O-phospho-Tyr, nitro-Tyr, Lys, Arg, ornithine, homo-Lys, homoarginine, nor-Lys, N-methyl-Lys, N,N′-dimethyl-Lys, N,N′,N′′-trimethyl-Lys or any syntheti
  PEPTIDE (8)..(9): basic amino acid; Xaa at residue 9 is Leu, Phe, Tyr, meta-Tyr, ortho-Tyr, nor-Tyr, mono-halo-Tyr, di-halo-Tyr, O-sulpho-Tyr, O-pho spho-Tyr, nitro-Tyr, Trp (D or L),  neo-Trp, halo-Trp (D or L) or any synthetic aromatic amino acid;
  PEPTIDE (11)..(11): Xaa at residue 11 is Pro, Hyp, Gly, an aliphatic amino acids bearing linear or branched saturated hydrocarbon chains such as Leu (D or L), Ile and Val or non-natural derivatives of the aliphatic amino acid;
  PEPTIDE (12)..(12): Xaa at residue 12 is Thr, Ser, g-Thr, g-Ser, Ala, an aliphatic amino acids bearing linear or branched saturated hydrocarbon chains such as Leu (D or L), Ile and Val or non-natural derivatives of the aliphatic amino acid, Phe, Tyr
  PEPTIDE (12)..(13): meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa at residue 13 is Pro, Hyp, Ser, Thr, g-Hyp,
  PEPTIDE (13)..(14): g-Ser, g-Thr or any hydroxylated amino acid; Xaa at residue 14 is an aliphatic amino acids bearing linear or branched saturated hydrocarbon chains such as Leu (D or L), Ile and Val or non-natural derivatives of the aliphatic amino acid,
  PEPTIDE (14)..(14): Phe, Tyr, meta-Tyr, ortho-Tyr, nor-Tyr, mono-halo-Tyr, di-halo-Tyr, O-sulpho-Tyr, O-phospho-Tyr, nitro-Tyr, Lys, Arg, ornithine, homo-Lys, homoarginine, nor-Lys, N-methyl-Lys, N,N′-dimethyl-Lys, N,N′,N′′-trimethyl-Lys or any syntheti
  PEPTIDE (14)..(15): basic amino acid; Xaa at residue 15 is Pro, Hyp, an aliphatic amino acids bearing linear or branched saturated hydrocarbon chains such as Leu (D or L), Ile and Val or non-natural derivatives of the aliphatic amino acid, Lys, Arg,
  PEPTIDE (15)..(16): ornithine, homo-Lys, homoarginine, nor-Lys, N-methyl-Lys, N,N′-dimethyl-Lys, N,N′,N′′-trimethyl-Lys or any synthetic basic amino acid; Xaa at residue 16 is Gly, His, Lys, Arg, ornithine, homo-Lys, homoarginine, nor-Lys,
  PEPTIDE (16)..(17): N-methyl-Lys, N,N′-dimethyl-Lys, N,N′, N′′-trimethyl-Lys or any synthetic basic amino acid; Xaa at residue 17 is des-Xaa15, Ser, Thr, g-Ser, g-Thr, Val, Asn, Phe, Tyr, meta-Tyr, ortho-Tyr, nor-Tyr, mono-halo-Tyr, di-halo-Tyr,
  PEPTIDE (17)..(18): O-sulpho-Tyr, O-phospho-Tyr, nitro-Tyr, Trp (D or L),  neo-Trp, halo-Trp (D or L) or any synthetic aromatic amino acid; Xaa at residue 18 is Met, Nle, an aliphatic amino acids bearing linear or branched saturated hydrocarbon chain
  PEPTIDE (18)..(18): such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L),  neo-Trp,
  PEPTIDE (18)..(19): halo-Trp (D or L), any synthetic aromatic amino acid, Arg, Lys, ornithine, homo-Lys, homoarginine, nor-Lys, N-methyl-Lys, N,N′-dimethyl-Lys, N,N′,N′′-trimethyl-Lys or any synthetic basic amino acid; Xaa at residue 19 is Pro, Hyp, Ser,
  PEPTIDE (19)..(19): Thr, g-Hyp, g-Ser, g-Thr, any hydroxylated amino acid, Ala, Met, Leu, Glu, Gla, Gln, Asp, Asn, any synthetic acidic amino acid, Tyr, meta-Tyr, ortho-Tyr, nor-Tyr, mono-halo-Tyr, di-halo-Tyr, O-sulpho-Tyr, O-phospho-Tyr, nitro-Tyr, His or Gly;
  PEPTIDE (22)..(24): Xaa at residue 22 is Gly, Ala, Ser, Thr, Pro, Hyp, g-Ser, g-Thr, g-Hyp, Glu, Asn or Gln; Xaa at residue 23 is Leu, Gly, Asn, Trp (D or L), neo-Trp or halo-Trp (D or L); Xaa at residue 24 is des-Xaa, Leu or Trp (D or L),  neo-Trp or halo-Trp (D or L);
  PEPTIDE (25)..(27): Xaa at residue 25 is des-Xaa or an aliphatic amino acids bearing linear or branched saturated hydrocarbon chains such as Leu (D or L), Ile and Val or non-natural derivatives of the aliphatic amino acid; Xaa at residue 27 is des-Xaa, Gly,
  PEPTIDE (27)..(28): Met, Nle, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa at residue 28 is des-Xaa, Pro,
  PEPTIDE (28)..(28): Hyp, Ala, an aliphatic amino acids bearing linear or branched saturated hydrocarbon chains such as Leu? (D or L), Ile and Val or non-natural derivatives of the aliphatic amino acid, Phe, Tyr, meta-Tyr, ortho-Tyr, nor-Tyr,
  PEPTIDE (28)..(29): mono-halo-Tyr, di-halo-Tyr, O-sulpho-Tyr, O-phospho-Tyr, nitro-Tyr, Trp (D or L),  neo-Trp, halo-Trp (D or L) or any synthetic aromatic amino acid; Xaa at residue 29 is Ala, an aliphatic amino acid bearing linear or branched
  PEPTIDE (29)..(29): saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr
  PEPTIDE (29)..(30): Trp (D or L),  neo-Trp, halo-Trp (D or L) or any synthetic aromatic amino acid; Xaa at residue 30 is Ala, an aliphatic amino acid bearing linear or branched saturated hydrocarbon chains such as Leu (D or L), Ile and Val or
  PEPTIDE (30)..(32): non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr or Phe; Xaa at residue 32 is an aliphatic amino acids bearing linear or branched
  PEPTIDE (32)..(33): saturated hydrocarbon chains such as Leu (D or L), Ile and Val or non-natural derivatives of the aliphatic amino acid; Xaa at resi ude 33 is des-Xaa, Asp, Glu, Gla, Pro, Hyp, Ser, Thr, g-Hyp, g-Se r, g-Ser or any synthetic hydroxylated amino acid
  PEPTIDE (34)..(34): Xaa at residue 34 is des-Xaa, Glu, Gla, Gln, Asp, Asn, any synthetic acidic amino acid, Lys, Arg, ornithine, homo-Lys, homoarginine, nor-Lys, N-methyl-Lys, N,N′-dimethyl-Lys, N,N′,N′′-trimethyl-Lys, any synthetic basic amino acid,
  PEPTIDE (34)..(36): Ile, Ser, Thr, g-Ser or g-Thr;  Xaa at residue 35 is des-Xaa, Pro, Hyp, Tyr, meta-Tyr, ortho-Tyr, nor-Tyr, mono-halo-Tyr, di-halo-Tyr, O-sulpho-Tyr, O-phospho-Tyr or nitro-Tyr;  Xaa at residue 36 is des-Xaa or Ph
XXCXXXXXXCXXXXXXXXXCCXXXXCXXXXCXXXXX

SEQ ID NO: 2 - protein, 31 aa, Conus magus
  PEPTIDE (1)..(31): Xaa at residue 3 may be Arg or Ser; Xaa at residues 12, 21 and 27 5 may be Pro or hydroxy-Pro; Xaa at residue 14 may be Ile or Leu ; Xaa at residue 17 may be Ile or Va
ACXKKWETCIVXIXGFXTCCXGLICGXFVCV

SEQ ID NO: 3 - protein, 31 aa, Conus skinneri
  PEPTIDE (1)..(31): Xaa at residue 5 is Lys, N-methyl-Lys, N,N-dimethyl-Lys or N,N,N-trimethyl-Lys; Xaa at residue 6, 16 and 18 may be Tyr, mono-halo-Tyr, di-halo-Tyr, O-sulpho-Tyr, O-phospho-Tyr or nitro-Ty
  PEPTIDE (1)..(31): Xaa at residue 7 may be Glu or gamma-carboxy-Glu; Xaa at residues 12 and 27 may be Pro or hydroxy-Pro
ACRQXXXFCLVXIIGXIXCCAGLICGXFVCL

SEQ ID NO: 4 - protein, 36 aa, Conus tessulatus
  PEPTIDE (1)..(36): Xaa at residue 1 may be Glu or gamma-carboxy-Glu; Xaa at residues 5, 8 and 34 may be Lys, N-methyl-Lys, N,N-dimethyl-Lys or N,N,N-trimethyl-Ly
  PEPTIDE (1)..(36): Xaa at residues 1, 33 and 35 may be Pro or hydroxy-Pro ; Xaa at residue 19 may be Tyr, mono-halo-Tyr, di-halo-Tyr, O-sulpho-Tyr, O-phospho-Tyr or nitro-Ty
XTCLXQDXFCIIXLIGTLXCCSGLICGFFVCVXXXF

SEQ ID NO: 5 - protein, 32 aa, Conus caracteristicus
  PEPTIDE (1)..(32): Xaa at residues 3 and 31 may be Glu or gamma-carboxy-Glu; Xaa at residues 12 and 30 may be Pro or hydroxy-Pro; Xaa at residue 15 may be Lys, N-methyl-Lys, N,N-dimethyl-Lys or N,N,N-trimethyl-Ly
  PEPTIDE (1)..(32): Xaa at residue 14 may be Trp or bromo-Trp; Xaa at residue 32 may be Tyr, mono-halo-Tyr, di-halo-Tyr, O-sulpho-Tyr, O-phospho-Tyr or nitro-Ty
DCXADGAFCGIXIVXNXMCCSNLCIFACVXXX

SEQ ID NO: 6 - protein, 31 aa, Conus textile
  PEPTIDE (1)..(31): Xaa at residues 4 and 21 is Glu or gamma-carboxy-Glu; Xaa at residues 6 and 8 is Trp or halo-Trp; Xaa at residue 10 is Pro or hydroxy-Pro; Xaa at residue 18 is Tyr, mono-halo-Tyr, di-halo-Tyr,
  PEPTIDE (1)..(31): O-sulpho-Tyr, O-phospho-Tyr or nitro-Tyr
DCHXRXDXCXASILGVIXCCXGLICFIAFCI

SEQ ID NO: 7 - protein, 33 aa, Conus textile
  PEPTIDE (1)..(33): Xaa at residues 4 and 7 is Glu or gamma-carboxy-Glu; Xaa at residue 5 isLys, N-methy-Lys, N,N-dimethyl-Lys or N,N,N-trimethyl-Lys; Xaa at residue 6 is Trp or halo-Trp; Xaa at residues 12, 21 and 2
  PEPTIDE (1)..(33): is Pro or hydroxy-Pro; Xaa at residue 18 isTyr, mono-halo-Tyr, di-halo-Tyr, O-sulpho-Tyr, O-phospho-Tyr or nitro-Ty
DCQXXXXFCIVXILGFVXCCXGLICGXFVCVDI

SEQ ID NO: 8 - protein, 31 aa, Conus tessulatus
  PEPTIDE (1)..(31): Xaa at residues 1 and 13 is Pro or hydroxy-Pro; Xaa at residues 6 and 18 is Tyr, mono-halo-Tyr, di-halo-Tyr, O-sulpho-Tyr, O-phospho-Tyr or nitro-Ty
XTCVSXNVFCGVXLVGTXLCCSGLVCLVVCI

SEQ ID NO: 9 - protein, 27 aa, Conus atlanticus
  PEPTIDE (1)..(27): Xaa at residues 3, 11, 13 and 15 is Pro or hydroxy-Pro; Xaa at residue 12 is  Lys, N-methy-Lys, N,N-dimethyl-Lys or N,N,N-trimethy l-Lys; Xaa at residue 16 is Tyr, mono-halo-Tyr, di-halo-Tyr
  PEPTIDE (1)..(27): O-sulpho-Tyr, O-phospho-Tyr or nitro-Tyr; Xaa at residue 21 is Trp or halo-Tr
CRXRGMFCGFXXXGXXCCNGXCFFVCI

SEQ ID NO: 10 - protein, 30 aa, Conus tessulatus
  PEPTIDE (1)..(30): Xaa at residue 2 is Trp or halo-Trp; Xaa at resude 8 is Glu or gamma-carboxy-Glu; Xaa 13 is Pro or hydroxy-Pr
RXCALDGXLCIIXVIGSIFCCHGICMIXCV

SEQ ID NO: 11 - protein, 30 aa, Conus arenatus
  PEPTIDE (1)..(30): Xaa at residues 4 and 12 is Pro or hydroxy-Pro; Xaa at residues 8 and 13 is Tyr, mono-halo-Tyr, di-halo-Tyr, O-sulpho-Tyr, O-phospho-Tyr or nitro-Tyr; Xaa at residue 14 is Lys, N-methy-Lys
  PEPTIDE (1)..(30): N,N-dimethyl-Lys or N,N,N-trimethyl-Lys; Xaa at residue 17 is Trp or halo-Tr
DCRXVGQXCGIXXXHNXRCCSQLCAIICVS

SEQ ID NO: 12 - protein, 30 aa, Conus generalis
  PEPTIDE (1)..(30): Xaa at residues 5 and 12 is Pro or hydroxy-Pro; Xaa at residues 7 and 17 isTyr, mono-halo-Tyr, di-halo-Tyr, O-sulpho-Tyr, O-phospho-Tyr or nitro-Tyr; Xaa at residue 29 is Glu or gamma-carboxy-Glu
GCLDXGXFCGTXFLGAXCCGGICLIVCIXT

SEQ ID NO: 13 - protein, 32 aa, Conus caracteristicus; identical to SEQ ID NO: 5
  PEPTIDE (1)..(32): Xaa at residues 3 and 31 may be Glu or gamma-carboxy-Glu; Xaa at residues 12 and 30 may be Pro or hydroxy-Pro; Xaa at residue 15 may be Lys, N-methyl-Lys, N,N-dimethyl-Lys or N,N,N-trimethyl-Ly
  PEPTIDE (1)..(32): Xaa at residue 14 may be Trp or bromo-Trp; Xaa at residue 32 may be Tyr, mono-halo-Tyr, di-halo-Tyr, O-sulpho-Tyr, O-phospho-Tyr or nitro-Ty

SEQ ID NO: 14 - protein, 32 aa, Conus caracteristicus
  PEPTIDE (1)..(32): Xaa at residues 3 and 31 may be Glu or gamma-carboxy-Glu; Xaa at residues 12 and 30 may be Pro or hydroxy-Pro; Xaa at residue 15 may be Lys, N-methyl-Lys, N,N-dimethyl-Lys or N,N,N-trimethyl-Ly
  PEPTIDE (1)..(32): Xaa at residue 14 may be Trp or bromo-Trp; Xaa at residue 32 may be Tyr, mono-halo-Tyr, di-halo-Tyr, O-sulpho-Tyr, O-phospho-Tyr or nitro-Ty
DCXADGAFCGIXTVXNXMCCSNLCIFACVXXX

SEQ ID NO: 15 - protein, 31 aa, Conus episcopatus
  PEPTIDE (1)..(31): Xaa at residues 4, 5 and 7 may be Glu or gamma-carboxy-Glu; Xaa at residue 6 may be Trp or bromo-Trp; Xaa at residues 12, 21 and 27 may be Pro or hydroxy-Pro; Xaa at residue 18 may be Tyr, mono-halo-Tyr,
  PEPTIDE (1)..(31): di-halo-Tyr, O-sulpho-Tyr, O-phospho-Tyr or nitro-Tyr
DCGXXXXFCIVXILGFVXCCXGLICGXFVCV

SEQ ID NO: 16 - protein, 31 aa, Conus nobilis; identical to SEQ ID NO: 3
  PEPTIDE (1)..(31): Xaa at residue 7 may be Glu or gamma-carboxy-Glu; Xaa at residue 5 may be Lys, N-methyl-Lys, N,N-dimethyl-Lys or N,N,N-trimethyl-Lys; Xaa at residue 6, 16 and 18 may be Tyr, mono-halo-Tyr, di-halo-Tyr, O-sulpho-Tyr, O-phospho-Tyr o
  PEPTIDE (1)..(31): nitro-Tyr; Xaa at residues 12 and 27 may be Pro or hydroxy-Pro

SEQ ID NO: 17 - protein, 36 aa, Conus tessulatus; identical to SEQ ID NO: 4
  PEPTIDE (1)..(36): Xaa at residues 1, 13, 33 and 35 may be Pro or hydroxy-Pro; Xaa at residue 5, 8 and 34 may be Lys, N-methyl-Lys, N,N-dimethyl-Lys or N,N,N-trimethyl-Lys; Xaa at residue 6 and 19 may be Tyr, mono-halo-Tyr, di-halo-Tyr,
  PEPTIDE (1)..(36): O-sulpho-Tyr, O-phospho-Tyr or nitro-Tyr

SEQ ID NO: 18 - protein, 31 aa, Conus tessulatus; identical to SEQ ID NO: 8
  PEPTIDE (1)..(31): Xaa at residues 1 and 13 is Pro or hydroxy-Pro; Xaa at residues 6 and 18 is Tyr, mono-halo-Tyr, di-halo-Tyr, O-sulpho-Tyr, O-phosp ho-Tyr or nitro-Ty

SEQ ID NO: 19 - protein, 31 aa, Conus textile; identical to SEQ ID NO: 6
  PEPTIDE (1)..(31): Xaa at residues 4 and 21 is Glu or gamma-carboxy-Glu; Xaa at residues 6 and 8 is Trp or halo-Trp; Xaa at residue 10 is Pro or hydroxy-Pro; Xaa at residue 18 is Tyr, mono-halo-Tyr, di-halo-Tyr,
  PEPTIDE (1)..(31): O-sulpho-Tyr, O-phospho-Tyr or nitro-Tyr

SEQ ID NO: 20 - protein, 33 aa, Conus textile; identical to SEQ ID NO: 7
  PEPTIDE (1)..(33): Xaa at residues 4 and 7 is Glu or gamma-carboxy-Glu; Xaa at residue 5 isLys, N-methy-Lys, N,N-dimethyl-Lys or N,N,N-trimethyl-Lys; Xaa at residue 6 is Trp or halo-Trp; Xaa at residues 12, 21 and 2
  PEPTIDE (1)..(33): is Pro or hydroxy-Pro; Xaa at residue 18 is Tyr, mono-halo-Tyr, di-halo-Tyr, O-sulpho-Tyr, O-phospho-Tyr or nitro-Ty

SEQ ID NO: 21 - DNA, 29 bp, Artificial Sequence
  amplification primer
caggatccatgaaactgacgtgyrtggtg

SEQ ID NO: 22 - DNA, 29 bp, Artificial Sequence
  amplification primer
atctcgagcacaggtatggatgactcagg

SEQ ID NO: 23 - DNA, 424 bp, Conus skinneri
  CDS (1)..(264)
atgaaactgacgtgtgtggtgatcgttgctgtgctgttcttgaccgcctggacattcgtcatggctgatgaccccagagatggagcggagattagaagcatggtaaggggggaacctctgtcgaaggcacgtgacgaaatgaaccccgaagcctctaaattggagaaaagggcgtgccgccaaaaatacgaattttgtctagtaccgatcattggatacatatattgctgcgctggcttaatctgtggtcctttcgtctgcctttgatagtgatgtcttctactgccatctgtgctacccctggcttgatctttgataggcgttgttgcccttcactggtttatgaaccctctgatcatactctctggacccttgggggtccaacatccaaataaagcgacatcccaaaaaaaaaaaaaaaaaa

SEQ ID NO: 24 - protein, 88 aa, Conus skinneri
MKLTCVVIVAVLFLTAWTFVMADDPRDGAEIRSMVRGEPLSKARDEMNPEASKLEKRACRQKYEFCLVPIIGYIYCCAGLICGPFVCL

SEQ ID NO: 25 - DNA, 418 bp, Conus tessulatus
  CDS (1)..(261)
ggatccatgaaactgacgtgcatggtgatcgttgttgtgctgttgttgaacgcctggacattcgtctccataaatggaaaggcgaatcgtttttggaaggcacgtgacgaaatgaaggactccgaagtttctgaattggagaaaaggaggaaaccgacctgcctgaagcaggacaagttttgcataataccgctcattggaaccctttattgctgcagtgggttaatctgtgggttttttgtctgcgtcccaaagccgttctgatgtcttctactgccatctgtgctacccctggcttgatctttgattggcgtgtgcccttcactggttatgaacccctctgatcctactgtctggacgcctcgggcgtccaacgtccaaataaagcgacatcccaataaaaaaaaaaaaaaaaaaa

SEQ ID NO: 26 - protein, 87 aa, Conus tessulatus
GSMKLTCMVIVVVLLLNAWTFVSINGKANRFWKARDEMKDSEVSELEKRRKPTCLKQDKFCIIPLIGTLYCCSGLICGFFVCVPKPF

SEQ ID NO: 27 - DNA, 280 bp, Conus caracteristicus
  CDS (1)..(249)
atgaaactgacgtgcgtgatgatcgttgctgtgctgttcttgaccgcctggacattcgtcacggctgatgactccattaatgcactggaggatcttttttcgaaggcacgtgacgaaatggaaaacggcgaagcttctacattgaacgagagagactgcgaagcagatggtgcattttgtggtatcccaattgtgaagaactggatgtgctgcagtaacttgtgtatttttgcctgcgtacccgagtattaagactgccgtgatgtcttctcctcccctc

SEQ ID NO: 28 - protein, 83 aa, Conus caracteristicus
MKLTCVMIVAVLFLTAWTFVTADDSINALEDLFSKARDEMENGEASTLNERDCEADGAFCGIPIVKNWMCCSNLCIFACVPEY

SEQ ID NO: 29 - DNA, 132 bp, Conus textile
  CDS (2)..(106)
attggagaaaagggattgccacgaaaggtgggattggtgtccagcatcaatccttggagtgatatattgctgcgagggattaatttgttttattgccttctgcatttgatagtgatgtcttctcctcccctc

SEQ ID NO: 30 - protein, 35 aa, Conus textile
LEKRDCHERWDWCPASILGVIYCCEGLICFIAFCI

SEQ ID NO: 31 - DNA, 132 bp, Conus textile
  CDS (2)..(112)
attggagaaaagggattgccaagagaaatgggagttttgtatagtaccgatccttggatttgtatattgctgccctggcttaatctgtggcccttttgtctgcgttgatatctgatgtcttctcctcccatc

SEQ ID NO: 32 - protein, 37 aa, Conus textile
LEKRDCQEKWEFCIVPILGFVYCCPGLICGPFVCVDI

SEQ ID NO: 33 - DNA, 288 bp, Conus tessulatus
  CDS (7)..(246)
ggatccatgaaactgacgtgtgtggtgatcgttgttgtgctgttgttgaacgcctggacattcgtctccataaatggaaaggcgaatcctttttggaaggcacgtgacgaaatgaaggactccgaagtttctgagttggagaaaaggaggaaaccgacctgcgtgtcgtataacgtgttttgcggagtaccgctcgttggaacctacctttgctgcagtggcttagtctgtctcgtagtctgcatctagtactgatgtcttctactcccatctgtgctacccctcgag

SEQ ID NO: 34 - protein, 80 aa, Conus tessulatus
MKLTCVVIVVVLLLNAWTFVSINGKANPFWKARDEMKDSEVSELEKRRKPTCVSYNVFCGVPLVGTYLCCSGLVCLVVCI

SEQ ID NO: 35 - DNA, 287 bp, Conus atlanticus
  CDS (7)..(240)
ggatccatgaaactgacgtgcgtggtgatcgttgctgtgctgttcttgaccgcctggacattcgtcacggctgatgactccataaatgggttggagaatctttttccgaaggcacgtcacgaaatgaggaaacccgaagcctctagatcgagagggaggtgccgtcctcgtggtatgttctgtggctttccgaaacctggaccatactgctgcaatggctggtgctttttcgtctgcatctaaaactgccgtgatgtgttctactcccatctgtgctacccctcgag

SEQ ID NO: 36 - protein, 78 aa, Conus atlanticus
MKLTCVVIVAVLFLTAWTFVTADDSINGLENLFPKARHEMRKPEASRSRGRCRPRGMFCGFPKPGPYCCNGWCFFVCI

SEQ ID NO: 37 - DNA, 289 bp, Conus tessulatus
  CDS (7)..(249)
ggatccatgaaactgacgtgcgtggtggtcgttgctgtgctgttcttgaacgcctggacattcgccacggctgttgactccaaacatgcactggcgaaactttttatgaaggcacgtgacgaaatgtataaccccgatgccactaaattggacgataagagatggtgcgctttagatggtgaactttgtatcataccggtcattgggtccatattttgctgccatggcatatgtatgatctactgcgtctagttgaactgccgtgatgtcttctactcccctctgtgct

SEQ ID NO: 38 - protein, 81 aa, Conus tessulatus
MKLTCVVVVAVLFLNAWTFATAVDSKHALAKLFMKARDEMYNPDATKLDDKRWCALDGELCIIPVIGSIFCCHGICMIYCV

SEQ ID NO: 39 - DNA, 340 bp, Conus arenatus
  CDS (7)..(246)
  misc_feature (1)..(340): n may be any nucleotide
ggatccatgaaactgacgtgtgtggtgatcgttgctgtgctgttcttgaccgcctggacattcgtcacggctgactccatacgtgcactggaggatttttttgcgaaggcacgtgacgaaatggaaaacagcggagcttctccattgaacgagagagactgccgacctgtaggtcaatattgtggcataccgtataagcacaactggcgatgctgcagtcagctttgtgcaattatctgtgtttcctaacccctctgatcctactctctgaagacctccgggattcaacatccaaataaagcgacatcccgatnaaaaaaaangaaaaaaaaaaaaaaaa

SEQ ID NO: 40 - protein, 80 aa, Conus arenatus
  misc_feature (1)..(340): n may be any nucleotide
MKLTCVVIVAVLFLTAWTFVTADSIRALEDFFAKARDEMENSGASPLNERDCRPVGQYCGIPYKHNWRCCSQLCAIICVS

SEQ ID NO: 41 - DNA, 293 bp, Conus generalis
  CDS (7)..(249)
ggatccatgaaactgacgtgtgtggtgatcgttgctgtgctattcttgaccgcctggacattcgtcacggctgatgacaccagatataaactggagaatccttttctgaaggcacgcaacgaactgcagaaacacgaagcctctcaactgaacgagagaggctgccttgacccaggttacttctgtgggacgccgtttcttggagcatactgctgcggtggcatttgccttattgtctgcatagaaacgtaaaggcttgatgtcttctactcccatctgtgctacccctcgag

SEQ ID NO: 42 - protein, 81 aa, Conus generalis
MKLTCVVIVAVLFLTAWTFVTADDTRYKLENPFLKARNELQKHEASQLNERGCLDPGYFCGTPFLGAYCCGGICLIVCIET

SEQ ID NO: 43 - DNA, 280 bp, Conus caracteristicus; identical to SEQ ID NO: 27

SEQ ID NO: 44 - protein, 83 aa, Conus caracteristicus; identical to SEQ ID NO: 28

SEQ ID NO: 45 - protein, 32 aa, Conus caracteristicus
  PEPTIDE (1)..(32): Xaa at residue 3 and 31is Glu or gamma-carboxy Glu; Xaa at residue 12 and 30 is Pro or Hydroxy Pro; Xaa at residue 17 is Trp or Bromo Trp; Xaa at residue 32 is Tyr, 125I-Tyr, Mono-Iodo Tyr, Di-Iodo Tyr, O-sulpho-Tyr or O-Phospho-Ty
DCXADGAFCGIXIVKNXMCCSNLCIFACVXXX

SEQ ID NO: 46 - DNA, 424 bp, Conus nobilis; identical to SEQ ID NO: 23

SEQ ID NO: 47 - protein, 88 aa, Conus nobilis; identical to SEQ ID NO: 24

SEQ ID NO: 48 - protein, 31 aa, Conus nobilis
  PEPTIDE (1)..(31): Xaa at residue 7 31is Glu or gamma-carboxy Glu; Xaa at residue 12 and 27 is Pro or Hydroxy Pro; Xaa at residue 6, 16 and 18 is Tyr, 125I-Tyr, Mono-Iodo Tyr, Di-Iodo Tyr, O-sulpho-Tyr or O-Phospho-Ty
ACRQKXXFCLVXIIGXIXCCAGLICGXFVCL

SEQ ID NO: 49 - DNA, 132 bp, Conus textile; identical to SEQ ID NO: 29

SEQ ID NO: 50 - protein, 35 aa, Conus textile; identical to SEQ ID NO: 30

SEQ ID NO: 51 - protein, 31 aa, Conus textile; identical to SEQ ID NO: 6
  PEPTIDE (1)..(31): Xaa at residue 4 and 21 is Glu or gamma-carboxy Glu; Xaa at residue 10 is Pro or Hydroxy Pro; Xaa at residue 6 and 8 is Trp or Bromo Trp; Xaa at residue 18 is Tyr, 125I-Tyr, Mono-Iodo Tyr, Di-Iodo Tyr, O-sulpho-Tyr or O-Phospho-Ty

SEQ ID NO: 52 - DNA, 132 bp, Conus textile; identical to SEQ ID NO: 31

SEQ ID NO: 53 - protein, 37 aa, Conus textile; identical to SEQ ID NO: 32

SEQ ID NO: 54 - protein, 33 aa, Conus textile
  PEPTIDE (1)..(33): Xaa at residue 4 and 7 is Glu or gamma-carboxy Glu; Xaa at residue 12, 21 and 27 is Pro or Hydroxy Pro; Xaa at residue 6 is Trp or Bromo Trp; Xaa at residue 18 is Tyr, 125I-Tyr, Mono-Iodo Tyr, Di-Iodo Tyr, O-sulpho-Tyr or O-Phospho-Ty
DCQXKXXFCIVXILGFVXCCXGLICGXFVCVDI

SEQ ID NO: 55 - DNA, 418 bp, Conus tessulatus; identical to SEQ ID NO: 25

SEQ ID NO: 56 - protein, 85 aa, Conus tessulatus
MKLTCMVIVVVLLLNAWTFVSINGKANRFWKARDEMKDSEVSELEKRRKPTCLKQDKFCIIPLIGTLYCCSGLICGFFVCVPKPF

SEQ ID NO: 57 - protein, 36 aa, Conus tessulatus
  PEPTIDE (1)..(36): Xaa at residue 1, 13, 33 and 35 is Pro or Hydroxy Pro; Xaa at residue 19 is Tyr, 125I-Tyr, Mono-Iodo Tyr, Di-Iodo Tyr, O-sulpho-Tyr or O-Phospho-Ty
XTCLKQDKFCIIXLIGTLXCCSGLICGFFVCVXKXF

SEQ ID NO: 58 - DNA, 288 bp, Conus tessulatus; identical to SEQ ID NO: 33

SEQ ID NO: 59 - protein, 80 aa, Conus tessulatus; identical to SEQ ID NO: 34

SEQ ID NO: 60 - protein, 31 aa, Conus tessulatus; identical to SEQ ID NO: 8
  PEPTIDE (1)..(31): Xaa at residue 1 and 13 is Pro or Hydroxy Pro; Xaa at residue 6 and 18 is Tyr, 125I-Tyr, Mono-Iodo Tyr, Di-Iodo Tyr, O-sulpho-Tyr or O-Phospho-Ty

SEQ ID NO: 61 - DNA, 357 bp, Conus caracteristicus
ggatccatgaaactgacgtgcatggtgatcgttgctgtgctgttcttgaccgcctggacattcgtcacggctgatgactccattaatgcactggaggatcttttttcgaaggcacgcgacgaaatggaaaacggcgaagcttctacattgaacgagagagactgcgaagcagatggtgcattttgtggtatcccaactgtgaagaactggatgtgctgcagtaacttgtgtatttttgcctgcgtacccgagtattaagactgccgtgatgtcttctgctcccctctgtgctacctggcttgatctttgattggcgcgtgcccttcactggttatgaacccctctgatcctactctc

SEQ ID NO: 62 - protein, 83 aa, Conus caracteristicus
MKLTCMVIVAVLFLTAWTFVTADDSINALEDLFSKARDEMENGEASTLNERDCEADGAFCGIPTVKNWMCCSNLCIFACVPEY

SEQ ID NO: 63 - protein, 32 aa, Conus caracteristicus
  PEPTIDE (1)..(32): Xaa at residue 3 and 31 is Glu or gamma-carboxy Glu; Xaa at residue 12 and 30 is Pro or Hydroxy Pro; Xaa at residue 17 is Trp or Bromo Trp; Xaa at residue 32 is Tyr, 125I-Tyr, Mono-Iodo Tyr, Di-Iodo Tyr, O-sulpho-Tyr or O-Phospho-Ty
DCXADGAFCGIXTVKNXMCCSNLCIFACVXXX

SEQ ID NO: 64 - DNA, 409 bp, Conus episcopatus
  misc_feature (1)..(409): n may be any nucleotide
ggatccatgaaactgacgtgtgtggtgatcattgctgtgctgttcttgaccgcctggacattcgtcatggctgatgactccaacaatggactggcgaatcatttttcgaaatcacgtgacgaaatggaggaccccgaagcttctaaattggagaaaagaagggattgccaagaagaatgggaattttgtatagtaccgatccttggattcgtatattgctgccctggcttaatctgtggtcctttcgtctgcgtttaatactgatgtcttctactcccatctgtgctacacctggcttgatctttgataggcgtgtgcccttcactggttataaacccctctgatcctactctctggacgcctcgggggcccaacatccaaataaagcaacatcccaatgaanaaaaaa

SEQ ID NO: 65 - protein, 83 aa, Conus episcopatus
MKLTCVVIIAVLFLTAWTFVMADDSNNGLANHFSKSRDEMEDPEASKLEKRRDCQEEWEFCIVPILGFVYCCPGLICGPFVCV

SEQ ID NO: 66 - protein, 31 aa, Conus episcopatus
  PEPTIDE (1)..(31): Xaa at residue 4, 5 and 7 is Glu or gamma-carboxy Glu; Xaa at residue 12, 21 and 27 is Pro or Hydroxy Pro; Xaa at residue 6 is Trp or Bromo Trp; Xaa at residue 18 is Tyr, 125I-Tyr, Mono-Iodo Tyr, Di-Iodo Tyr, O-sulpho-Tyr or O-Phospho-Ty
DCQXXXXFCIVXILGFVXCCXGLICGXFVCV

(19) United States
(12) Patent Application Publication
(10) Pub. No.: US 2002/0198145 A1
(43) Pub. Date: Dec. 26, 2002

(54) MuO-conopeptides and their use as local anesthetics

(75) Inventors: Baldomero M. Olivera, Salt Lake City, UT (US); J. Michael McIntosh, Salt Lake City, UT (US); R. Tyler McCabe, Salt Lake City, UT (US); James E. Garrett, Salt Lake City, UT (US); Richard T. Layer, Sandy, UT (US); John D. Wagstaff, Salt Lake City, UT (US); Robert M. Jones, Salt Lake City, UT (US)

Correspondence Address: ROTHWELL, FIGG, ERNST & MANBECK, P.C., 555 13TH STREET, N.W., SUITE 701, EAST TOWER, WASHINGTON, DC, 20004, US

(73) Assignee: COGNETIX, INC., Salt Lake City, UT (US)

(21) Appl. No.: 09/908,739

(22) Filed: Jul. 20, 2001

Related U.S. Application Data
(63) Continuation-in-part of application No. 09/590,386, filed on Jun. 9, 2000.
(60) Provisional application No. 60/138,507, filed on Jun. 10, 1999.
(60) Provisional application No. 60/219,451, filed on Jul. 20, 2000.

(51) Int. Cl. A61K 38/17

(52) U.S. Cl. 514/12

(57) ABSTRACT

The present invention is directed to the new μO-conopeptides, their coding sequences and their propeptides and to the use of μO-conopeptides as a local anesthetic for treating pain. The μO-conopeptides have long lasting anesthetic activity and are particularly useful for spinal anesthesia, either administered acutely for post-operative pain or via an intrathecal pump for severe chronic pain situations or for treatment of pain in epithelial tissue.

### CROSS-REFERENCE TO RELATED APPLICATIONS

The present application is a continuation-in-part of Ser. No. 09/590,386 filed on Jun. 9, 2000 and claims benefit thereto. The present application also claims benefit under 35 USC §119(e) to U.S. provisional patent applications Ser. No. 60/138,507 filed on Jun. 10, 1999 and Ser. No. 60/219,451 filed on Jul. 20, 2000. Each of these applications is incorporated herein by reference.

This invention was made with Government support under Grant No. GM48677 awarded by the National Institute of General Medical Sciences, National Institutes of Health, Bethesda, Md. The United States Government has certain rights in the invention.

### BACKGROUND OF THE INVENTION

The present invention is directed to the use of μO-conopeptides as a local anesthetic for treating pain. The μO-conopeptides have long-lasting anesthetic activity and are particularly useful for spinal anesthesia, administered either acutely for post-operative pain or via an intrathecal pump for severe chronic pain situations. The present invention is further directed to new μO-conopeptides, their coding sequences and their propeptides.

The publications and other materials used herein to illuminate the background of the invention, and in particular, cases to provide additional details respecting the practice, are incorporated herein by reference, and for convenience, are referenced by author and date in the following text and respectively grouped in the appended Bibliography.

Conus is a genus of predatory marine gastropods (snails) which envenomate their prey. Venomous cone snails use a highly developed projectile apparatus to deliver their cocktail of toxic conotoxins into their prey. In fish-eating species such as Conus magus the cone detects the presence of the fish using chemosensors in its siphon and when close enough extends its proboscis and fires a hollow harpoon-like tooth containing venom into the fish. This immobilizes the fish and enables the cone snail to wind it into its mouth via an attached filament. For general information on Conus and their venom see the website address http://grimwade.biochem.unimelb.edu.au/cone/referenc.html. Prey capture is accomplished through a sophisticated arsenal of peptides which target specific ion channel and receptor subtypes. Each Conus species venom appears to contain a unique set of 50-200 peptides. The composition of the venom differs greatly between species and between individual snails within each species, each optimally evolved to paralyse it's prey. The active components of the venom are small peptides toxins, typically 12-30 amino acid residues in length and are typically highly constrained peptides due to their high density of disulphide bonds.

The venoms consist of a large number of different peptide components that when separated exhibit a range of biological activities: when injected into mice they elicit a range of physiological responses from shaking to depression. The paralytic components of the venom that have been the focus of recent investigation are the α-, ω- and μ-conotoxins. All of these conotoxins act by preventing neuronal communication, but each targets a different aspect of the process to achieve this. The α-conotoxins target nicotinic ligand gated channels, the μ-conotoxins target the voltage-gated sodium channels and the ω-conotoxins target the voltage-gated calcium channels (Olivera et al., 1985). For example a linkage has been established between α-, αA- & φ-conotoxins and the nicotinic ligand-gated ion channel; ω-conotoxins and the voltage-gated calcium channel; μ-conotoxins and the voltage-gated sodium channel; δ-conotoxins and the voltage-gated sodium channel; κ-conotoxins and the voltage-gated potassium channel; conantokins and the ligand-gated glutamate (NMDA) channel. For a partial list of Conus peptides and their amino acid sequences see the website address http://pir.georgetown.edu.

However, the structure and function of only a small minority of these peptides have been determined to date. For peptides where function has been determined, three classes of targets have been elucidated: voltage-gated ion channels; ligand-gated ion channels, and G-protein-linked receptors.

Conus peptides which target voltage-gated ion channels include those that delay the inactivation of sodium channels, as well as blockers specific for sodium channels, calcium channels and potassium channels. Peptides that target ligand-gated ion channels include antagonists of NMDA and serotonin receptors, as well as competitive and noncompetitive nicotinic receptor antagonists. Peptides which act on G-protein receptors include neurotensin and vasopressin receptor agonists. The unprecedented pharmaceutical selectivity of conotoxins is at least in part defined by a specific disulfide bond frameworks combined with hypervariable amino acids within disulfide loops (for a review see McIntosh et al., 1998).

The pain response is a protective reflex system warning an individual of hostile situations and tissue injury. The origins of clinically significant acute and chronic pain in a mammal are different, but the biochemical and neurological pathways are similar. In the following discussion on pain and its management, the focus is primarily on humans, however, it should be understood that the concepts of pain are applicable to mammalian animals and the management of such pain is applicable to veterinary medicine.

Acute pain is often associated with surgery and with trauma. The intensity of acute postoperative pain varies considerably depending on the extent of the surgical procedure performed, on the individual's pain sensitivity, and on the type of anesthetic management employed during surgery. In general, major operations on the thorax and the upper abdominal region induce the most intensive postoperative pain. Extensive orthopedic operations also produce strong postoperative pain.

Chronic pain is persistent pain which has long outlasted the onset of any known or suspected physical cause. It can occur after a known injury or disease, or it can occur without any known physical cause whatsoever. Moreover, it can be accompanied by known tissue pathology, such as chronic inflammation that occurs in some types of arthritis, or it can occur long after the healing of the injured tissue which is suspected or known to be the cause of chronic pain. Chronic pain is a very general concept and there are several varieties of chronic pain related to the musculoskeletal system, visceral organs, skin, and nervous system.

Neuropathic pain can occur as a form of chronic pain and can also occur under acute conditions such as those following surgery or accidental trauma. Neuropathic pain can be defined as pain that results from an abnormal functioning of the peripheral and/or central nervous system. A critical component of this abnormal functioning is an exaggerated response of pain-related nerve cells either in the peripheral or in the central nervous system. This exaggerated responsiveness is manifested behaviorally as increased sensitivity to pain, i.e., as hyperalgesia or allodynia, both of which can occur in chronic neuropathic and acute inflammatory pains. An example is the pain from causalgia wherein even a light touch to the skin is felt as an excruciating burning pain (allodynia) or a normally mild pain is experienced as an excruciating one (hyperalgesia). Neuropathic pain is thought to be a consequence of damage to peripheral nerves or to regions of the central nervous system. However, abnormal functioning of pain-related regions of the nervous system call also occur with chronic inflammatory conditions such as certain types of arthritis and metabolic disorders such as diabetes as well as with acute inflammatory conditions. Thus, many types of chronic pains that are related to inflammation as well as acute pains that are related to inflammation can be considered to be at least partly neuropathic pains.

The modem concept of pain treatment emphasizes the significance of prophylactic prevention of pain, as pain is more easily prevented than relieved. Additionally, the hormonal stress responses associated with pain are considered harmful to the patient, impair the healing process and overall recovery, and generally are to be avoided.

While compounds utilized as general anesthetics reduce pain by producing a loss of consciousness, local anesthetics act to induce a loss of sensation in the localized area of administration in the body. The mechanism by which local anesthetics induce their effect, while not having been determined definitively, is generally thought to be based upon the ability to interfere with the initiation and transmission of the nerve impulse conduction along an axon through a reversible blockade of sodium channels. Currently used local anesthetics have durations of action lasting only several hours. While this length of duration meets many needs, particularly the control of acute pain, local anesthetic agents with longer duration of action would have broad clinical application for the treatment of postoperative and chronic pain (Kuzma et al., 1997).

The duration of action of a local anesthetics is proportional to the time during which it is in actual contact with the nervous tissues. In an effort to increase the duration of action, procedures or formulations that maintain localization of the drug at the nerve greatly prolong anesthesia. All local anesthetics are potentially toxic, and therefore it is of great importance that the choice of drug, concentration, rate and site of administration, as well as other actors, be considered in their use. On the other hand, a local anesthetic must remain at the site long enough to allow sufficient time for the localized pain to subside. Different devices and formulations are known in the art for administration of local anesthetics. See U.S. Pat. No. 5,747,060, which discloses such devices and formulations.

Side effects which have been associated with the use of different drugs for treating pain or as local anesthetics includes include respiratory depression, reduced cough reflex, bronchial spasms, nausea, vomiting, release of histamine, peripheral vasodilation, orthostatic hypotension, vagal impact on the heart, contraction of smooth muscles (sphincters), reduced peristaltic motility in the gastrointestinal tract, urinary retention, stimulated release of adrenalin, anti-diuretic hormone, changes in the regulation of body temperature and sleep pattern, tolerance, addiction, tachycardia, increase in blood pressure, and agitation. Not all of these side effects are seen with any given drug used to treat pain.

Thus, there is a need to develop additional drugs and methods which can be used for the treatment of pain, which can act as local anesthetics, which have a longer duration of action and which have reduced side effects. Accordingly, an object of the invention is to provide methods and compositions for the treatment of acute or chronic pain which provide effective control of pain with longer duration of action and reduced side effects associated with traditional analgesics.

### SUMMARY OF THE INVENTION

The present invention is directed to the new μO-conopeptides, their coding sequences and their propeptides and to the use of μO-conopeptides as a local anesthetic for treating pain. The μO-conopeptides have long lasting anesthetic activity and are particularly useful for spinal anesthesia, either administered acutely for post-operative pain or via an intrathecal pump for severe chronic pain situations or for treatment of pain in epithelial tissue.

More specifically, the present invention is directed to μO-conopeptides having the general formula I:

Xaa₁-Xaa₂-Cys-Xaa₃-Xaa₄-Xaa₅-Xaa₆-Xaa₇-Xaa₈-Cys-Xaa₉-Xaa₁₀-Xaa₁₁-Xaa₁₂-Xaa₁₃-Xaa₁₄-Xaa₁₅-Xaa₁₆-Xaa₁₇-Cys-Cys-Xaa₁₈-Xaa₁₉-Xaa₂₀-Xaa₂₁-Cys-Xaa₂₂-Xaa₂₃-Xaa₂₄-Xaa₂₅-CYS-Xaa₂₆-Xaa₂₇-Xaa₂₈-Xaa₂₉-Xaa₃₀ (SEQ ID NO:1),

wherein Xaa₁ is des-Xaa₁, Pro, hydroxy-Pro (Hyp), Arg, Lys, ornithine, homo-Lys, homoarginine, nor-Lys, N-methyl-Lys, N,N′-dimethyl-Lys, N,N′,N″-trimethyl-Lys or any synthetic basic amino acid; Xaa₂ is des-Xaa₂, Ala, Gly, Asp, Glu, γ-carboxy-glutamate (Gla), any synthetic acidic amino acid, Thr, Ser, g-Thr (where g is glycosylation), g-Ser, Trp (D or L), neo-Trp or halo-Trp (D or L) or Xaa₂ may be pyroglutamate if Xaa₁ is des-Xaa₁; Xaa₃ is Arg, Lys, ornithine, homo-Lys, homoarginine, nor-Lys, N-methyl-Lys, N,N′-dimethyl-Lys, N,N′,N″-trimethyl-Lys, any synthetic basic amino acid, Ser, Thr, g-Ser, g-Thr, Ala, an aliphatic amino acid bearing linear or branched saturated hydrocarbon chains such as Leu (D or L), Ile and Val or non-natural derivatives of the aliphatic amino acid, His, Glu, Gln, Gla, Asp, Asn or any synthetic acidic amino acid; Xaa₄ is Glu, Gla, Gln, Asp, Asn, any synthetic acidic amino acid, Lys, Arg, ornithine, homo-Lys, homoarginine, nor-Lys, N-methyl-Lys, N,N′-dimethyl-Lys, N,N′,N″-trimethyl-Lys, any synthetic basic amino acid, Ala, an aliphatic amino acid bearing linear or branched saturated hydrocarbon chains such as Leu (D or L), Ile and Val or non-natural derivatives of the aliphatic amino acid, Ser, Thr, Pro, Hyp, g-Ser, g-Thr, g-Hyp or any synthetic hydroxylated amino acid; Xaa₅ is Lys, Arg, ornithine, homo-Lys, homoarginine, nor-Lys, N-methyl-Lys, N,N′-dimethyl-Lys, N,N′,N″-trimethyl-Lys, any synthetic basic amino acid, Tyr, meta-Tyr, ortho-Tyr, nor-Tyr, mono-halo-Tyr, di-halo-Tyr, O-sulpho-Tyr, O-phospho-Tyr, nitro-Tyr, an aliphatic amino acid bearing linear or branched saturated hydrocarbon chains such as Leu (D or L), Ile and Val or non-natural derivatives of the aliphatic amino acid, Glu, Gla, Gln, Asp, Asn, any synthetic acidic amino acid, Pro or Hyp; Xaa₆ is Trp (D or L), neo-Trp, halo-Trp (D or L), Gly, Tyr, meta-Tyr, ortho-Tyr, nor-Tyr, mono-halo-Tyr, di-halo-Tyr, O-sulpho-Tyr, O-phospho-Tyr, nitro-Tyr, Glu, Gla, Gln, Asp, Asn, any synthetic acidic amino acid; Xaa₇ is Glu, Gla, Gin, Asp, Asn, any synthetic acidic amino acid, Met, norleucine (Nle), Ala, an aliphatic amino acid bearing linear or branched saturated hydrocarbon chains such as Leu (D or L), Ile and Val or non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr, Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa₈ is Leu, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₉ is Pro, Hyp, Gly, an aliphatic amino acid bearing linear or branched saturated hydrocarbon chains such as Leu (D or L), Ile and Val or non-natural derivatives of the aliphatic amino acid; Xaa₁₀ is Thr, Ser, g-Thr, g-Ser, Ala, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₁₁ is Pro, Hyp, Ser, Thr, g-Hyp, g-Ser, g-Thr or any hydroxylated amino acid; Xaa₁₂ is an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa₁₃ is Pro, Hyp, an aliphatic amino acid bearing linear or branched saturated hydrocarbon chains such as Leu (D or L), Ile and Val or non-natural derivatives of the aliphatic amino acid, Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa₁₄ is Gly, His, Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa₁₅ is des-Xaa₁₅, Ser, Thr, g-Ser, g-Thr, Val, Asn, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₁₆ is Met, Nle, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Tip (D or L), any synthetic aromatic amino acid, Arg, Lys, ornithine, homo-Lys, homoarginine, nor-Lys, N-methyl-Lys, N,N′-dimethyl-Lys, N,N′,N″-trimethyl-Lys or any synthetic basic amino acid; Xaa₁₇ is Pro, Hyp, Ser, Thr, g-Hyp, g-Ser, g-Thr, any hydroxylated amino acid, Ala, Met, Leu, Glu, Gla, Gln, Asp, Asn, any synthetic acidic amino acid, Tyr, meta-Tyr, ortho-Tyr, nor-Tyr, mono-halo-Tyr, di-halo-Tyr, O-sulpho-Tyr, O-phospho-Tyr, nitro-Tyr, His or Gly; Xaa₁₈ is Gly, Ala, Ser, Thr, Pro, Hyp, g-Ser, g-Thr, g-Hyp, Glu, Asn or Gln; Xaa₁₉ is Leu, Gly, Asn, Trp (D or L), neo-Trp or halo-Trp (D or L); Xaa₂₀ is des-Xaa₂₀, Leu or Trp (D or L), neo-Trp or halo-Trp (D or L); Xaa₂₁ is des-Xaa₂₁ or an aliphatic amino acid bearing linear or branched saturated hydrocarbon chains such as Leu (D or L), Ile and Val or non-natural derivatives of the aliphatic amino acid; Xaa₂₂ is des-Xaa₂₂, Gly, Met, Nle, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₂₃ is des-Xaa₂₃, Pro, Hyp, Ala, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₂₄ is Ala, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₂₅ is Ala, an aliphatic amino acid bearing linear or branched saturated hydrocarbon chains such as Leu (D or L), Ile and Val or non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr or Phe; Xaa₂₆ is an aliphatic amino acid bearing linear or branched saturated hydrocarbon chains such as Leu (D or L), Ile and Val or non-natural derivatives of the aliphatic amino acid; Xaa₂₇ is des-Xaa₂₇, Asp, Glu, Gla, Pro, Hyp, Ser, Thr, g-Hyp, g-Ser, g-Ser or any synthetic hydroxylated amino acid; Xaa₂₈ is des-Xaa₂₈, Glu, Gla, Gln, Asp, Asn, any synthetic acidic amino acid, Lys, Arg, ornithine, homo-Lys, homoarginine, nor-Lys, N-methyl-Lys, N,N′-dimethyl-Lys, N,N′,N″-trimethyl-Lys, any synthetic basic amino acid, Ile, Ser, Thr, g-Ser or g-Thr; Xaa₂₉ is des-Xaa₂₉, Pro, Hyp, Tyr, meta-Tyr, ortho-Tyr, nor-Tyr, mono-halo-Tyr, di-halo-Tyr, O-sulpho-Tyr, O-phospho-Tyr or nitro-Tyr; Xaa₃₀ is des-Xaa₃₀ or Phe, with the proviso that the peptide is not MrVIA/B as defined below. The Cys residues may be in D or L configuration and may optionally be substituted with homocysteine (D or L). The Tyr residues may be substituted with ¹²⁵I-Tyr or with the 3-hydroxyl or 2-hydroxyl isomers and corresponding O-sulpho- and O-phospho-derivatives. The acidic amino acid residues may be substituted with any synthetic acidic amino acid, e.g., tetrazolyl derivatives of Gly and Ala. The nonnatural derivatives of the aliphatic amino acids include those synthetic derivatives bearing non-natural aliphatic branched or linear side chairs CₙH₂ₙ₊₂ up to and including n=8. The halogen is iodo, chloro, fluoro or bromo; preferably iodo for halogen substituted-Tyr and bromo for halogen-substituted Trp.

MrVIA/B has the sequence: Ala-Cys-Xaa₃₁-Lys-Lys-Trp-Glu-Tyr-Cys-Ile-Val-Xaa₃₂-Ile-Xaa₃₃-Gly-Phe-Xaa₃₄-Tyr-Cys-Cys-Xaa₃₂-Gly-Leu-Ile-Cys-Gly-Xaa₃₂-Phe-Val-Cys-Val, wherein Xaa₃₁ is Arg or Ser, Xaa₃₂ is Pro or hydroxy-Pro, Xaa₃₃ is Ile or Leu and Xaa₃₄ is lie or Val (SEQ ID NO:2).

The present invention is also directed to novel specific conotoxin peptides within general formula I having the formulas:

Ala-Cys-Arg-Gln-Xaa₁-Xaa₂-Xaa₃-Phe-Cys-Leu-Val-Xaa₄-Ile-Ile-Gly-Xaa₂-Ile-Xaa₂-Cys-Cys-Ala-Gly-Leu-Ile-Cys-Gly-Xaa₄-Phe-Val-Cys-Leu (SEQ ID NO:3);

Xaa₄-Thr-Cys-Leu-Xaa₁-Gln-Asp-Xaa₁-Phe-Cys-Ile-Ile-Xaa₄-Leu-Ile-Gly-Thr-Leu-Xaa₂-Cys-Cys-Ser-Gly-Leu-Ile-Cys-Gly-Phe-Phe-Val-Cys-Val-Xaa₄-Xaa₁-Xaa₄-Phe (SEQ ID NO:4);

Asp-Cys-Xaa₃-Ala-Asp-Gly-Ala-Phe-Cys-Gly-Ile-Xaa₄-Ile-Val-Xaa₁-Asn-Xaa₅-Met-Cys-Cys-Ser-Asn-Leu-Cys-Ile-Phe-Ala-Cys-Val-Xaa₄-Xaa₃-Xaa₂ (SEQ ID NO:5);

Asp-Cys-His-Xaa₃-Arg-Xaa₅-Asp-Xaa₅-Cys-Xaa₄-Ala-Ser-Ile-Leu-Gly-Val-Ile-Xaa₂-Cys-Cys-Xaa₃-Gly-Leu-Ile-Cys-Phe-Ile-Ala-Phe-Cys-Ile (SEQ ID NO:6);

Asp-Cys-Gln-Xaa₃-Xaa₁-Xaa₅-Xaa₃-Phe-Cys-Ile-Val-Xaa₄-Ile-Leu-Gly-Phe-Val-Xaa₂-Cys-Cys-Xaa₄-Gly-Leu-Ile-Cys-Gly-Xaa₄-Phe-Val-Cys-Val-Asp-Ile (SEQ ID NO:7);

Xaa₄-Thr-Cys-Val-Ser-Xaa₂-Asn-Val-Phe-Cys-Gly-Val-Xaa₄-Leu-Val-Gly-Thr-Xaa₂-Leu-Cys-Cys-Ser-Gly-Leu-Val-Cys-Leu-Val-Val-Cys-Ile (SEQ ID NO:8);

Cys-Arg-Xaa₄-Arg-Gly-Met-Phe-Cys-Gly-Phe-Xaa₄-Xaa₄-Xaa₄-Gly-Xaa₄-Xaa₂-Cys-Cys-Asn-Gly-Xaa₅-Cys-Phe-Phe-Val-Cys-Ile (SEQ ID NO:9);

Arg-Xaa₅-Cys-Ala-Leu-Asp-Gly-Xaa₃-Leu-Cys-Ile-Ile-Xaa₄-Val-Ile-Gly-Ser-Ile-Phe-Cys-Cys-His-Gly-Ile-Cys-Met-Ile-Xaa₂-Cys-Val (SEQ ID NO:10);

Asp-Cys-Arg-Xaa₄-Val-Gly-Gln-Xaa₂-Cys-Gly-Ile-Xaa₄-Xaa₂-Xaa₁-His-Asn-Xaa₅-Arg-Cys-Cys-Ser-Gln-Leu-Cys-Ala-Ile-Ile-Cys-Val-Ser (SEQ ID NO:11);

Gly-Cys-Leu-Asp-Xaa₄-Gly-Xaa₂-Phe-Cys-Gly-Thr-Xaa₄-Phe-Leu-Gly-Ala-Xaa₂-Cys-Cys-Gly-Gly-Ile-Cys-Leu-Ile-Val-Cys-Ile-Xaa₃-Thr (SEQ ID NO:12),

Asp-Cys-Xaa₃-Ala-Asp-Gly-Ala-Phe-Cys-Gly-Ile-Xaa₄-Ile-Val-Xaa₁-Asn-Xaa₅-Met-Cys-Cys-Ser-Asn-Leu-Cys-Ile-Phe-Ala-Cys-Val-Xaa₄-Xaa₃-Xaa₂ (SEQ ID NO:13);

Asp-Cys-Xaa₃-Ala-Asp-Gly-Ala-Phe-Cys-Gly-Ile-Xaa₄-Thr-Val-Xaa₁-Asn-Xaa₅-Met-Cys-Cys-Ser-Asn-Leu-Cys-Ile-Phe-Ala-Cys-Val-Xaa₄-Xaa₃-Xaa₂ (SEQ ID NO:14);

Asp-Cys-Gln-Xaa₃-Xaa₃-Xaa₅-Xaa₃-Phe-Cys-Ile-Val-Xaa₄-Ile-Leu-Gly-Phe-Val-Xaa₂-Cys-Cys-Xaa₄-Gly-Leu-Ile-Cys-Gly-Xaa₄-Phe-Val-Cys-Val (SEQ ID NO:15);

Ala-Cys-Arg-Gln-Xaa₁-Xaa₂-Xaa₃-Phe-Cys-Leu-Val-Xaa₄-Ile-Ile-Gly-Xaa₂-Ile-Xaa₂-Cys-Cys-Ala-Gly-Leu-Ile-Cys-Gly-Xaa₄-Phe-Val-Cys-Leu (SEQ ID NO:16);

Xaa₄-Thr-Cys-Leu-Xaa₁-Gln-Asp-Xaa₁-Phe-Cys-Ile-Ile-Xaa₄-Leu-Ile-Gly-Thr-Leu-Xaa₂-Cys-Cys-Ser-Gly-Leu-Ile-Cys-Gly-Phe-Phe-Val-Cys-Val-Xaa₄-Xaa₁-Xaa₄-Phe (SEQ ID NO:17);

Xaa₄-Thr-Cys-Val-Ser-Xaa₂-Asn-Val-Phe-Cys-Gly-Val-Xaa₄-Leu-Val-Gly-Thr-Xaa₂-Leu-Cys-Cys-Ser-Gly-Leu-Val-Cys-Leu-Val-Val-Cys-Ile (SEQ ID NO:18);

Asp-Cys-His-Xaa₃-Arg-Xaa₅-Asp-Xaa₅-Cys-Xaa₄-Ala-Ser-Ile-Leu-Gly-Val-Ile-Xaa₂-Cys-Cys-Xaa₃-Gly-Leu-Ile-Cys-Phe-Ile-Ala-Phe-Cys-Ile (SEQ ID NO:19); and

Asp-Cys-Gln-Xaa₃-Xaa₁-Xaa₅-Xaa₃-Phe-Cys-Ile-Val-Xaa₄-Ile -Leu-Gly-Phe-Val-Xaa₂-Cys-Cys-Xaa₄-Gly-Leu-Ile-Cys-Gly-Xaa₄-Phe-Val-Cys-Val-Asp-Ile (SEQ ID NO:20),

wherein Xaa₁ is Lys, N-methy-Lys, N,N-dimethyl-Lys or N,N,N-trimethyl-Lys; Xaa₂ is Tyr, mono-halo-Tyr, di-halo-Tyr, O-sulpho-Tyr, O-phospho-Tyr or nitro-Tyr; Xaa₃ is Glu or gamma-carboxy-Glu (Gla); Xaa₄ is Pro or hydroxy-Pro; Xaa₅ is Trp or halo-Trp; and the C-terminus contains a carboxyl or amide group. The halo is preferably chlorine or iodine, more preferably iodine. In addition, the Arg residues may be substituted by Lys, ornithine, homoargine, nor-Lys, N-methyl-Lys, N,N-dimethyl-Lys, N,N,N-trimethyl-Lys or any synthetic basic amino acid; the Xaa₁ residues may be substituted by Arg, ornithine, homoargine, nor-Lys, or any synthetic basic amino acid; the Tyr residues may be substituted with any synthetic hydroxy containing amino acid; the Ser residues may be substituted with Thr or any synthetic hydroxylated amino acid; the Thr residues may be substituted with Ser or any synthetic hydroxylated amino acid; the Phe and Trp residues may be substituted with any synthetic aromatic amino acid; and the Asn, Ser, Thr or Hyp residues may be glycosylated. The Cys residues may be in D or L configuration and may optionally be substituted with homocysteine (D or L). The Tyr residues may also be substituted with ¹²⁵I-Tyr or with the 3-hydroxyl or 2-hydroxyl isomers (meta-Tyr or ortho-Tyr, respectively) and corresponding O-sulpho-and O-phospho-derivatives. The acidic amino acid residues may be substituted with any synthetic acidic amino acid, e.g., tetrazolyl derivatives of Gly and Ala. The aliphatic amino acids may be substituted by synthetic derivatives bearing non-natural aliphatic branched or linear side chains CₙH₂ₙ₊₂ up to and including n=8.

More specifically, the present invention is directed to the following μO-conopeptides within general formula I:

MrVIA: SEQ ID NO:2, wherein Xaa₃₀ is Arg, Xaa₃₁ is Ile and Xaa₃₂ is Ile;

MrVIB: SEQ ID NO:2, wherein Xaa₃₀ is Ser, Xaa₃₁ is Leu and Xaa₃₂ is Val;

A657: SEQ ID NO:3, wherein Xaa₁ is Lys, Xaa₂ is Tyr, Xaa₃ is Glu and Xaa₄ is Pro;

F079: SEQ ID NO:4, wherein Xaa₁ is Lys, Xaa₂ is Tyr and Xaa₄ is Pro;

Ca6.1: SEQ ID NO:5, wherein Xaa₁ is Lys, Xaa₂ is Tyr, Xaa₃ is Glu, Xaa₄ is Pro and Xaa₅ is Trp;

Tx6.12: SEQ ID NO:6, wherein Xaa₂ is Tyr, Xaa₃ is Glu, Xaa₄ is Pro and Xaa₅ is Trp;

Tx6.13: SEQ ID NO:7, wherein Xaa₁ is Lys, Xaa₂ is Tyr, Xaa₃ is Glu, Xaa₄ is Pro and Xaa₅ is Trp;

G28: SEQ ID NO:8, wherein Xaa₂ is Tyr and Xaa₄ is Pro;

F763: SEQ ID NO:9, wherein Xaa₁ is Lys, Xaa₂ is Tyr, Xaa₄ is Pro and Xaa₅ is Trp;

F080: SEQ ID NO:10, wherein Xaa₂ is Tyr, Xaa₃ is Glu, Xaa₄ is Pro and Xaa₅ is Trp;

F008: SEQ ID NO:11, wherein Xaa₁ is Lys, Xaa₂ is Tyr, Xaa₄ is Pro and Xaa₅ is Trp;

G18: SEQ ID NO:12, wherein Xaa₂ is Tyr, Xaa₃ is Glu and Xaa₄ is Pro;

Ca6.1: SEQ ID NO:13, wherein Xaa₁ is Lys, Xaa₂ is Tyr, Xaa₃ is Glu, Xaa₄ is Pro and Xaa₅ is Trp;

Ca6.2: SEQ ID NO:14, wherein Xaa₁ is Lys, Xaa₂ is Tyr, Xaa₃ is Glu, Xaa₄ is Pro and Xaa₅ is Trp;

Ep6.3: SEQ ID NO:15, wherein Xaa₂ is Tyr, Xaa₃ is Glu, Xaa₄ is Pro and Xaa₅ is Trp;

Nb6.1: SEQ ID NO:16, wherein Xaa₁ is Lys, Xaa₂ is Tyr, Xaa₃ is Glu and Xaa₄ is Pro;

Ts6.1: SEQ ID NO:17, wherein Xaa₁ is Lys, Xaa₂ is Tyr and Xaa₄ is Pro;

Ts6.5: SEQ ID NO:18, wherein Xaa₂ is Tyr and Xaa₄ is Pro;

Tx6.12: SEQ ID NO:19, wherein Xaa₂ is Tyr, Xaa₃ is Glu, Xaa₄ is Pro and Xaa₅ is Trp; and

Tx6.13: SEQ ID NO:20, wherein Xaa₁ is Lys, Xaa₂ is Tyr, Xaa₃ is Glu, Xaa₄ is Pro and Xaa₅ is Trp.

Examples of synthetic aromatic amino acid include, but are not limited to, such as nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acid, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also http://www.amino-acids.com), incorporated herein by reference, by and available from RSP Amino Acid Analogues, Inc., Worcester, Mass. Examples of synthetic acid amino acids include those derivatives bearing acidic functionality, including carboxyl, phosphate, sulfonate and synthetic tetrazolyl derivatives such as described by Ornstein et al. (1993) and in U.S. Pat. No. 5,331,001, each incorporated herein by reference.

Optionally, in the peptides of general formula I and the specific peptides described above, the Asn residues may be modified to contain an N-glycan and the Ser, Thr and Hyp residues may be modified to contain an O-glycan (e.g., g-N, g-S, g-T and g-Hyp). In accordance with the present invention, a glycan shall mean any N-, S- or O-linked mono-, di-, tri-, poly- or oligosaccharide that can be attached to any hydroxy, amino or thiol, group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The gylcan may also include similar polyhydroxy groups, such as D-penicillamine 2,5 and halogenated derivatives thereof or polypropylene glycol derivatives. The glycosidic linkage is beta and 1-4 or 1-3, preferably 1-3. The linkage between the glycan and the amino acid may be alpha or beta, preferably alpha and is 1-.

Core O-glycans have been described by Van de Steen et al. (1998), incorporated herein by reference. Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have been identified. The type of glycosidic linkage (orientation and connectivities) are defined for each core glycan. Suitable glycans and glycan analogs are described further in U.S. Ser. No. 09/420,797 filed Oct. 19, 1999 and in PCT Application No. PCT/US99/24380 filed Oct. 19, 1999 (PCT Published Application No. WO 00/23092), each incorporated herein by reference. A preferred glycan is Gal(β1→3)GalNAc(α1→).

Optionally, in the peptides of general formula I and the specific peptides described above, pairs of Cys residues may be replaced pairwise with isoteric lactam or ester-thioether replacements, such as Ser/(Glu or Asp), Lys/(Glu or Asp) or Cys/Ala combinations. Sequential coupling by known methods (Barnay et al., 2000; Hruby et al., 1994; Bitan et al., 1997) allows replacement of native Cys bridges with lactam bridges. Thioether analogs may be readily synthesized using halo-Ala residues commercially available from RSP Amino Acid Analogues.

The present invention is further directed to derivatives of the above peptides and peptide derivatives which are acylic permutations in which the cyclic permutants retain the native bridging pattern of native toxin. See Craik et al. (2001).

The present invention is also directed to the identification of the nucleic acid sequences encoding these peptides and their propeptides and the identication of nucleic acid sequences of additional related μO-conopeptides.

The present invention is further directed to a method of reducing/alleviating/decreasing the perception of pain by a subject or for inducing analgesia, particularly local analgesia, in a subject comprising administering to the subject an effective amount of the pharmaceutical composition comprising a therapeutically effective amount of a μO-conotoxin peptide described herein or a pharmaceutically acceptable salt or solvate thereof, including MrVIA and MrVIB. The present invention is also directed to a pharmaceutic al composition comprising a therapeutically effective amount of a μO-conotoxin peptide described herein or a pharmaceutically acceptable salt or solvate thereof and a pharmaceutically acceptable carrier.

### BRIEF DESCRIPTION OF THE FIGURES

FIG. 1 shows μO-conopeptide MrVIB inhibits skin flinch sensitivity in the Guinea pig intracutaneous wheal assay with greater potency than lidocaine or bupivacaine. Data represent the number of flinches observed after 36 pin pricks in a 30 minutes test period. Each point represents the mean of at least three observations.

FIG. 2 shows μO-conopeptide MrVIB produces a long-lasting inhibition of skin flinch sensitivity relative to either lidocaine or bupivacaine in the Guinea pig intracutaneous wheal assay. Data represent the percentage of flinches observed out of six total at each time point. Each point represents the mean of at least three observations.

### DETAILED DESCRIPTION OF THE INVENTION

The present invention is directed to the new μO-conopeptides, their coding sequences and their propeptides and to the use of μO-conopeptides as a local anesthetic for treating pain. The μO-conopeptides have long lasting anesthetic activity and are particularly useful for spinal anesthesia, either administered acutely for post-operative pain or via an intrathecal pump for severe chronic pain situations or for treatment of pain in epithelial tissue.

The present invention, in another aspect, relates to a pharmaceutical composition comprising an effective amount of a conotoxin peptide described herein or a pharmaceutically acceptable salt or solvate thereof. Such a pharmaceutical composition has the capability of acting as analgesic agents.

The present invention also provides for a method provides local anesthesia to a patient having pain. In one embodiment, the pain results from surgical or medical procedures, and the compounds are administered to the central nervous system (CNS), e.g. to the spine for spinal analgesia. In a second embodiment, the pain is in an epithelial tissue region associated with damage or loss of epithelial tissue as a result of, for example, plastic surgery, canker sores, burns, sore throats, genital lesions, upper or lower gastrointestinal bronchoscopy or endoscopy, intubation, dermatologic abrasions, scratched corneas or chemical skin peels, and the compounds are administered to alleviate the associated pain. The μO-conopeptides administered in accordance with the method of the invention are also beneficial in relieving pain of the corneas.

The conotoxin peptides described herein are sufficiently small to be chemically synthesized. General chemical syntheses for preparing the foregoing conotoxin peptides are described hereinafter. Various ones of the conotoxin peptides can also be obtained by isolation and purification from specific Conus species using the technique described in U.S. Pat. No. 4,447,356 (Olivera et al., 1984), the disclosure of which is incorporated herein by reference.

Although the conotoxin peptides of the present invention can be obtained by purification from cone snails, because the amounts of conotoxin peptides obtainable from individual snails are very small, the desired substantially pure conotoxin peptides are best practically obtained in commercially valuable amounts by chemical synthesis using solid-phase strategy. For example, the yield from a single cone snail may be about 10 micrograms or less of conotoxin peptide. By “substantially pure” is meant that the peptide is present in the substantial absence of other biological molecules of the same type; it is preferably present in an amount of at least about 85% purity and preferably at least about 95% purity. Chemical synthesis of biologically active conotoxin peptides depends of course upon correct determination of the amino acid sequence.

The conotoxin peptides can also be produced by recombinant DNA techniques well known in the art. Such techniques are described by Sambrook et al. (1989). The peptides produced in this manner are isolated, reduced if necessary, and oxidized to form the correct disulfide bonds.

One method of forming disulfide bonds in the peptides of the present invention is the air oxidation of the linear peptides for prolonged periods under cold room temperatures or at room temperature. This procedure results in the creation of a substantial amount of the bioactive, disulfide-linked peptides. The oxidized peptides are fractionated using reverse-phase high performance liquid chromatography (HPLC) or the like, to separate peptides having different linked configurations. Thereafter, either by comparing these fractions with the elution of the native material or by using a simple assay, the particular fraction having the correct linkage for maximum biological potency is easily determined. However, because of the dilution resulting from the presence of other fractions of less biopotency, a somewhat higher dosage may be required.

The peptides are synthesized by a suitable method, such as by exclusively solid-phase techniques, by partial solid-phase techniques, by fragment condensation or by classical solution couplings.

In conventional solution phase peptide synthesis, the peptide chain can be prepared by a series of coupling reactions in which constituent amino acids are added to the growing peptide chain in the desired sequence. Use of various coupling reagents, e.g., dicyclohexylcarbodiimide or diisopropylcarbonyldimidazole, various active esters, e.g., esters of N-hydroxyphthalimide or N-hydroxy-succinimide, and the various cleavage reagents, to carry out reaction in solution, with subsequent isolation and purification of intermediates, is well known classical peptide methodology. Classical solution synthesis is described in detail in the treatise, “Methoden der Organischen Chemie (Houben-Weyl): Synthese von Peptiden,” (1974). Techniques of exclusively solid-phase synthesis are set forth in the textbook, “Solid-Phase Peptide Synthesis,” (Stewart and Young, 1969), and are exemplified by the disclosure of U.S. Pat. No. 4,105,603 (Val, et al., 1978). The fragment condensation method of synthesis is exemplified in U.S. Pat. No. 3,972,859 (1976). Other available syntheses are exemplified by U.S. Pat. No. 3,842,067 (1974) and U.S. Pat. No. 3,862,925 (1975). The synthesis of peptides containing γ-carboxyglutamic acid residues is exemplified by Rivier et al. (1987), Nishiuchi et al. (1993) and Zhou et al. (1996).

Common to such chemical syntheses is the protection of the labile side chain groups of the various amino acid moieties with suitable protecting groups which will prevent a chemical reaction from occurring at that site until the group is ultimately removed. Usually also common is the protection of an α-amino group on an amino acid or a fragment while that entity reacts at the carboxyl group, followed by the selective removal of the α-amino protecting group to allow subsequent reaction to take place at that location. Accordingly, it is common that, as a step in such a synthesis, an intermediate compound is produced which includes each of the amino acid residues located in its desired sequence in the peptide chain with appropriate side-chain protecting groups linked to various ones of the residues having labile side chains.

As far as the selection of a side chain amino protecting group is concerned, generally one is chosen which is not removed during deprotection of the α-amino groups during the synthesis. However, for some amino acids, e.g., His, protection is not generally necessary. In selecting a particular side chain protecting group to be used in the synthesis of the peptides, the following general rules are followed: (a) the protecting group preferably retains its protecting properties and is not split off under coupling conditions, (b) the protecting group should be stable under the reaction conditions selected for removing the α-amino protecting group at each step of the synthesis, and (c) the side chain protecting group must be removable, upon the completion of the synthesis containing the desired amino acid sequence, under reaction conditions that will not undesirably alter the peptide chain.

It should be possible to prepare many, or even all, of these peptides using recombinant DNA technology. However, when peptides are not so prepared, they are preferably prepared using the Merrifield solid-phase synthesis, although other equivalent chemical syntheses known in the art can also be used as previously mentioned. Solid-phase synthesis is commenced from the C-terminus of the peptide by coupling a protected α-amino acid to a suitable resin. Such a starting material can be prepared by attaching an α-amino-protected amino acid by an ester linkage to a chloromethylated resin or a hydroxymethyl resin, or by an amid e bond to a benzhydrylamine (BHA) resin or paramethylbenzhydrylamine NBHA) resin. Preparation of the hydroxymethyl resin is described by Bodansky et al. (1966). Chloromethylated resins are commercially available from Bio Rad Laboratories (Richmnond, Calif.) and from Lab. Systems, Inc. The preparation of such a resin is described by Stewart and Young (1969). BHA and MBHA resin supports are commercially available, and are generally used when the desired polypeptide being synthesized has an unsubstituted amide at the C-terminus. Thus, solid resin supports may be any of those known in the art, such as one having the formulae —O—CH₂—resin support, —NH BHA resin support, or —NH—MBHA resin support. When the unsubstituted amide is desired, use of a BHA or MBHA resin is preferred, because cleavage directly gives the amide. In case the N-methyl amide is desired, it can be generated from an N-methyl BHA resin. Should other substituted amides be desired, the teaching of U.S. Pat. No. 4,569,967 (Kornreich et al., 1986) can be used, or should still other groups than the free acid be desired at the C-terminus, it may be preferable to synthesize the peptide using classical methods as set forth in the Houben-Weyl text (1974).

The C-terminal amino acid, protected by Boc or Fmoc and by a side-chain protecting group, if appropriate, can be first coupled to a chloromethylated resin according to the procedure set forth in Horiki et al. (1978), using KF in DMF at about 60° C. for 24 hours with stirring, when a peptide having free acid at the C-terminus is to be synthesized. Following the coupling of the BOC-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific α-amino protecting groups may be used as described in Schroder & Lubke (1965).

After removal of the α-amino-protecting group, the remaining α-amino- and side chain-protected amino acids are coupled step-wise in the desired order to obtain the intermediate compound defined hereinbefore, or as an alternative to adding each amino acid separately in the synthesis, some of them may be coupled to one another prior to addition to the solid phase reactor. Selection of an appropriate coupling reagent is within the skill of the art. Particularly suitable as a coupling reagent is N,N′-dicyclohexylcarbodiimide (DCC, DIC, HBTU, HATU, TBTU in the presence of HoBt or HoAt).

The activating reagents used in the solid phase synthesis of the peptides are well known in the peptide art. Examples of suitable activating reagents are carbodiimides, such as N,N′-diisopropylcarbodiimide and N-ethyl-N′-(3-dimethylaminopropyl)carbodiimide. Other activating reagents and their use in peptide coupling are described by Schroder & Lubke (1965) and Kapoor (1970).

Each protected amino acid or amino acid sequence is introduced into the solid-phase reactor in about a twofold or more excess, and the coupling may be carried out in a medium of dimethylformamide (DMF):CH₂Cl₂ (1:1) or in DMF or CH₂Cl₂ alone. In cases where intermediate coupling occurs, the coupling procedure is repeated before removal of the α-amino protecting group prior to the coupling of the next amino acid. The success of the coupling reaction at each stage of the synthesis, if performed manually, is preferably monitored by the ninhydrin reaction, as described by Kaiser et al. (1970). Coupling reactions can be performed automatically, as on a Beckman 990 automatic synthesizer, using a program such as that reported in Rivier et al. (1978).

After the desired amino acid sequence has been completed, the intermediate peptide can be removed from the resin support by treatment with a reagent, such as liquid hydrogen fluoride or TFA (if using Fmoc chemistry), which not only cleaves the peptide from the resin but also cleaves all remaining side chain protecting groups and also the α-amino protecting group at the N-terminus if it was not previously removed to obtain the peptide in the form of the free acid. If Met is present in the sequence, the Boc protecting group is preferably first removed using trifluoroacetic acid (TFA)/ethanedithiol prior to cleaving the peptide from the resin with HF to eliminate potential S-alkylation. When using hydrogen fluoride or TFA for cleaving, one or more scavengers such as anisole, cresol, dimethyl sulfide and methylethyl sulfide are included in the reaction vessel.

Cyclization of the linear peptide is preferably affected, as opposed to cyclizing the peptide while a part of the peptido-resin, to create bonds between Cys residues. To effect such a disulfide cyclizing linkage, fully protected peptide can be cleaved from a hydroxymethylated resin or a chloromethylated resin support by ammonolysis, as is well known in the art, to yield the fully protected amide intermediate, which is thereafter suitably cyclized and deprotected. Alternatively, deprotection, as well as cleavage of the peptide from the above resins or a benzhydrylamine (BHA) resin or a methylbenzhydrylamine (MBHA), can take place at 0° C. with hydrofluoric acid (HF) or TFA, followed by oxidation as described above.

The peptides are also synthesized using an automatic synthesizer. Amino acids are sequentially coupled to an MBHA Rink resin (typically 100 mg of resin) beginning at the C-terminus using an Advanced Chemtech 357 Automatic Peptide Synthesizer. Couplings are carried out using 1,3-diisopropylcarbodimide in N-methylpyrrolidinone (NMP) or by 2-(1H-benzotriazole-1-yl)-1,1,3,3-tetramethyluronium hexafluorophosphate (HBTU) and diethylisopro-pylethylamine (DIEA). The FMOC protecting group is removed by treatment with a 20% solution of piperidine in dimethylformamide(DMF). Resins are subsequently washed with DMF (twice), followed by methanol and NMP.

Additional conotoxin peptides are identified by cloning by reverse transcription-polymerase chain reaction (RT-PCR) from cone snail venom duct mRNA. The PCR primers are based on the DNA sequences coding for the precursor peptides of the “O-Superfamily” as described herein. RT-PCR of venom duct mRNA produces a product of about 250-300 nucleotides in Conus species that express conotoxin genes. The PCR product is then cloned into a plasmid vector and individual clones are sequenced to determine the sequence of various conotoxin genes. Alternatively, cDNA libraries are prepared from Conus venom duct using conventional techniques. DNA from single clones is amplified by conventional techniques using primers which correspond approximately to the M13 universal priming site and the M13 reverse universal priming site. Clones having a size of approximately 250 nucleotides are sequenced and screened for similarity in sequence to the propeptide described herein. In this manner, conotoxins having the basic structure and activity described herein are cloned from many Conus species.

Muteins, analogs or active fragments (collectively referred to herein as derivatives) of μO-conopeptides are also contemplated for use as local anesthetics. See, e.g., Hammerland et al. (1992). Derivative muteins, analogs or active fragments of μO-conopeptides may be synthesized according to known techniques, including conservative amino acid substitutions, such as outlined in U.S. Pat. Nos. 5,545,723; 5,534,615 and 5,364,769. The derivative muteins, analogs or active fragments may be conveniently assayed for activity by using a hindlimb paralysis test such as described in Example 2 or a local anesthetic test such as described in Example 3.

A variety of peptides from Conus target sodium channels. μ-Conopeptides (i.e., GVIA) block sodium channels expressed by muscle cells (Olivera et al., 1990). δ-Conopeptides (i.e., GmVIA) delay the inactivation of neuronal sodium channels (Olivera et al., 1990). Another class of conopeptide (i.e., μ-PnIVA and μ-PnIVB; unfortunately also called μ but having a distinct cysteine framework from that which is considered a μ-conopeptide) blocks sodium channels in molluscan neurons, but has no effect on sodium currents in bovine chromaffin cells or in rat brain synaptosomes (Fainzilber et al., 1995). Finally, the μO-conopeptides (MrVIA and MrVIB) block mammalian sodium channels (McIntosh et al., 1995).

Since the μO-conopeptides have been shown to have a slow and incomplete washout from Xenopus oocytes expressing cloned rat type II sodium channels (Terlau et al., 1996), the present invention examined whether the μO-conopeptides might represent a candidate for a long-lasting local anesthetic.

Thus, the present invention is directed to a method for inducing local analgesia by administering the μO-conopeptides described herein. In one embodiment, the pain results from surgical or medical procedures, and the compounds are administered to the central nervous system (CNS), e.g. to the spine for spinal analgesia. Thus, the μO-conopeptides administered in accordance with the method of the invention are beneficial for spinal anesthesia, administered either acutely for post-operative pain or via an intrathecal pump for severe chronic pain situations.

In a second embodiment, μO-conopeptides are used to provide local anesthesia for pain associated with any epithelial tissue region in a subject, for example, pain associated with epithelial ulcers, such as a canker sore or genital lesions. Canker sores can occur alone or in groups on the inside of the cheek or lip or underneath the tongue. Severely affected people have continuously recurring ulcers which last for one to two weeks (Clayman). Genital ulcers are usually caused by sexually transmitted diseases, including herpes and syphilis. The early stages of syphilis are characterized by a hard chancre, a painful ulcer where bacteria has penetrated the skin. This may be followed by shallow, elongated ulcers once the chancre has healed. Such ulcers are painful. Genital ulceration may also be a side effect of drugs taken orally or caused by solutions applied to genital warts. Pain in epithelial tissue is also caused by bums. Bums affecting the epidermal layer are usually associated with pain, restlessness and fever. Treatment of such a burn in accordance with the method of the invention can provide relief from the attendant pain. Pain as a result of damage to or loss of epithelial tissue is also associated with other conditions and procedures, such as sore throats and plastic surgery, for example carbon dioxide laser surgery to remove for skin resurfacing and removal of wrinkles (Rosenberg et al., 1996), burns, genital lesions, upper or lower gastrointestinal bronchoscopy or endoscopy, intubation, dermatologic abrasions, scratched corneas or chemical skin peels. The μO-conopeptides administered in accordance with the method of the invention are beneficial in relieving pain associated with such damaged tissues. The μO-conopeptides administered in accordance with the method of the invention are also beneficial in relieving pain of the corneas.

Pharmaceutical compositions containing a μO-conopeptide or pharmaceutically acceptable salts thereof as the active ingredient (agent) can be prepared according to conventional pharmaceutical compounding techniques. See, for example, Remington's Pharmaceutical Sciences, 18th Ed. (1990, Mack Publishing Co., Easton, Pa.). Typically, a therapeutically effective amount of the active ingredient will be admixed with a pharmaceutically acceptable carrier. The carrier may take a wide variety of forms depending on the form of preparation desired for administration, e.g., intravenous, oral or parenteral. The compositions may further contain antioxidizing agents, stabilizing agents, preservatives and the like.

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or EL quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as, sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

For topical administration, the compound may be formulated as an ointment, cream, gel, paste or solution comprising the compound to be administered in a pharmaceutical acceptable carrier. One means of topical administration is a transdermal patch containing the compound to be administered.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion.

For example, administration of the active agent according to this invention may be achieved using any suitable delivery means, including:

(a) pump (see, e.g., Lauer & Hatton (1993), Zimm et al. (1984) and Ettinger et al. (1978));

(b) microencapsulation (see, e.g., U.S. Pat. Nos. 4,352,883; 4,353,888; and 5,084,350);

(c) continuous release polymer implants (see, e.g., U.S. Pat. No. 4,883,666);

(d) macroencapsulation (see, e.g., U.S. Pat. Nos. 5,284,761, 5,158,881, 4,976,859 and 4,968,733 and published PCT patent applications WO92/19195, WO 95/05452);

(e) naked or unencapsulated cell grafts to the CNS (see, e.g., U.S. Pat. Nos. 5,082,670 and 5,618,531);

(f) injection, either subcutaneously, intravenously, intra-arterially, intramuscularly, or to other suitable site;

(g) oral administration, in capsule, liquid, tablet, pill, or prolonged release formulation; or

(h) topical (see, e.g., U.S. Pat. Nos. 6,046,187 and 6,030,974).

In one embodiment of this invention, an active agent is delivered directly into the CNS, preferably to the brain ventricles, brain parenchyma, the intrathecal space or other suitable CNS location, most preferably intrathecally.

Alternatively, targeting therapies may be used to deliver the active agent more specifically to certain types of cells, by the use of targeting systems such as antibodies or cell-specific ligands. Targeting may be desirable for a variety of reasons, e.g. if the agent is unacceptably toxic, if it would otherwise require too high a dosage, or if it would not otherwise be able to enter target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

The active agent is preferably administered in an therapeutically effective amount. By a “therapeutically effective amount” or simply “effective amount” of an active compound is meant a sufficient amount of the compound to treat or alleviate pain or to induce analgesia at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

For the treatment of pain, if the route of administration is directly to the CNS, the dosage contemplated is from about 1 ng to about 100 mg per day, preferably from about 100 ng to about 10 mg per day, more preferably from about 1 μg to about 100 jig per day. If administered peripherally, the dosage contemplated is somewhat higher, from about 100 ng to about 1000 mg per day, preferably from about 10 μg to about 100 mg per day, more preferably from about 100 μg to about 10 mg per day.

If the μO-conopeptide is delivered by continuous infusion (e.g., by pump delivery, biodegradable polymer delivery or cell-based delivery), then a lower dosage is contemplated than for bolus delivery.

However, it will be understood that the amount of the active compound actually administered will be determined by a physician, in the light of the relevant circumstances including the condition to be treated, the chosen route of administration, the age, weight, and response of the individual patient, and the severity of the patient's symptoms, and therefore the above dosage ranges are not intended to limit the scope of the invention in any way. As used herein the terms “pharmaceutical compositions” and “pharmaceutically acceptable” include compositions and ingredients for both human and veterinary use.

The present data suggest that μO-conopeptides are extremely potent and long-lasting local anesthetic agents, most likely due to their ability to block neuronal sodium channels. Moreover, since μO-conopeptides probably act at a site on sodium channels distinct from other local anesthetics or guanidinium toxins like tetrodotoxin (since they are likely to act at an extracellular target, but do compete for [³H]saxitoxin at site I) (Terlau et al., 1996), and probably do not affect sodium channels in the muscles or heart (since i.p. injection of 10 nmol is without effect in mice (McIntosh et al., 1995), these peptides lack the untoward side effects of clinically used local anesthetics.

Despite the high hydrophobicity of these peptides, there is a cluster of charged amino acid residues at the amino terminus. This cluster of charge, combined with the size of the peptides, probably results in poor permeation of the nerve sheath and thus accounts for the poor efficacy in the tail withdrawal assay. In contrast, when the nerve sheath is not a barrier, such as following intrathecal injection or intracutaneous injection, μO-conopeptides are effective and long-lasting. These facts establish that μO-conopeptides are novel candidates for spinal anesthesia, either administered acutely for post-operative pain or via an intrathecal pump for severe chronic pain situations.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982; Sambrook et al., 1989; Ausubel et al., 1992; Glover, 1985; Anand, 1992; Guthrie and Fink, 1991; Harlow and Lane, 1988; Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (]R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986).

### EXAMPLES

The present invention is further detailed in the following Examples, which are offered by way of illustration and are not intended to limit the invention in any manner. Standard techniques well known in the art or the techniques specifically described below are utilized.

### Example 1

### Isolation of μO-conopeptides A657 and F079

PCR primers designed to amplify “O Superfamily” conotoxin genes were used in RT-PCR amplification of venom duct cDNA from a variety of Conus species. The primers have the following sequences:

| | | |
|---|---|---|
| Forward Primer: μOCon6 | CAGGATCCATGAAACTGACGTGYRTGGTG | (SEQ ID NO:22) |
| Reverse Primer: μOCon7 | ATCTCGAGCACAGGTATGGATGACTCAGG | (SEQ ID NO:23). |

Amplification products in the appropriate size range were cloned and sequenced. A range of “O-Superfamily” gene sequences were identified. The novel genes, A657 from C. skinneri, F079, F080 and G28 from C. tessulatus, F763 from C. atlanticus, F008 from C. arenatus, Tx6.12 and Tx6.13 from C. textile and G18 from C. generalis, were identified as μO-conopeptides on the basis of their similarity to the μ-O conopeptides MrVIA and MrVIB. This similarity was much greater than the similarity with any of the ω-, κ- or δ-conopeptides that comprise the “O Superfamily” peptides. The cDNA and amino acid sequence for the A657, F079, Ca6.1, Tx6.12 (portion), Tx6.13 (portion), G28, F763, F080, F008 and G18 propeptides are set forth in Tables 1-10, respectively. The amino acid sequences of the mature μO-conopeptides are as shown above. The DNA and amino acid sequences for the Ca6.1, Ca6.2, Ep6.3, Nb6.1, Ts6.1, Ts6.5, Tx6.12 and Tx6.13 are shown in Table 11.

TABLE 1
+HZ,41
DNA Sequence (SEQ ID NO:23) and Protein
Sequence (SEQ ID NO:24) of A657

atg aaa ctg acg tgt gtg gtg atc gtt gct gtg ctg ttc ttg acc gcc
Met Lys Leu Thr Cys Val Val Ile Val Ala Val Leu Phe Leu Thr Ala
tgg aca ttc gtc atg gct gat gac ccc aga gat gga gcg gag att aga
Trp Thr Phe Val Met Ala Asp Asp Pro Arg Asp Gly Ala Glu Ile Arg
agc atg gta agg ggg gaa cct ctg tcg aag gca cgt gac gaa atg aac
Ser Met Val Arg Gly Glu Pro Leu Ser Lys Ala Arg Asp Glu Met Asn
ccc gaa gcc tct aaa ttg gag aaa agg gcg tgc cgc caa aaa tac gaa
Pro Glu Ala Ser Lys Leu Glu Lys Arg Ala Cys Arg Gln Lys Tyr Glu
ttt tgt cta gta ccg atc att gga tac ata tat tgc tgc gct ggc tta
Phe Cys Leu Val Pro Ile Ile Gly Tyr Ile Tye Cys Cys Ala Gly Leu
atc tgt ggt cct ttc gtc tgc ctt tgatagtgat gtcttctact gccatctgtg
Ile Cys Gly Pro Phe Val Cys Leu
ctacccctgg cttgatcttt gataggcgtt gttgcccttc actggtttat gaaccctctg
atcatactct ctggaccctt gggggtccaa catccaaata aagcgacatc ccaaaaaaaa
aaaaaaaaaa

TABLE 2
DNA Sequence (SEQ ID NO:25) and
Protein Sequence (SEQ ID NO:26) of F079

gga tcc atg aaa ctg acg tgc atg gtg atc gtt gtt gtg ctg ttg ttg
Gly Ser Met Lys Leu Thr Cys Met Val Ile Val Val Val Leu Leu Leu
aac gcc tgg aca ttc gtc tcc ata aat gga aag gcg aat cgt ttt tgg
Asn Ala Trp Thr Phe Val Ser Ile Asn Gly Lys Ala Asn Arg Phe Trp
aag gca cgt gac gaa atg aag gac tcc gaa gtt tct gaa ttg gag aaa
Lys Ala Arg Asp Glu Met Lys Asp Ser Glu Val Ser Glu Leu Glu Lys
agg agg aaa ccg acc tgc ctg aag cag gac aag ttt tgc ata ata ccg
Arg Arg Lys Pro Thr Cys Leu Lys Gln Asp Lys Phe Cys Ile Ile Pro
ctc att gga acc ctt tat tgc tgc agt ggg tta atc tgt ggg ttt ttt
Leu Ile Gly Thr Leu Tyr Cys Cys Ser Gly Leu Ile Cys Gly Phe Phe
gtc tgc gtc cca aag ccg ttc tgatgtcttc tactgccatc tgtgctaccc
Val Cys Val Pro Lys Pro Phe
ctggcttgat ctttgattgg cgtgtgccct tcactggtta tgaacccctc tgatcctact
gtctggacgc ctcgggcgtc caacgtccaa ataaagcgac atcccaataa aaaaaaaaaa
aaaaaaa

TABLE 3
DNA Sequence (SEQ ID NO:27) and Protein
Sequence (SEQ ID NO:28) of Ca6.1

atg aaa ctg acg tgc gtg atg atc gtt gct gtg ctg ttc ttg acc gcc
Met Lys Leu Thr Cys Val Met Ile Val Ala Val Leu Phe Leu Thr Ala
tgg aca ttc gtc acg gct gat gac tcc att aat gca ctg gag gat ctt
Trp Thr Phe Val Thr Ala Asp Asp Ser Ile Asn Ala Leu Glu Asp Leu
ttt tcg aag gca cgt gac gaa atg gaa aac ggc gaa gct tct aca ttg
Phe Ser Lys Ala Arg Asp Glu Met Clu Asn Gly Glu Ala Ser Thr Leu
aac gag aga gac tgc gaa gca gat ggt gca ttt tgt ggt atc cca att
Asn Glu Arg Asp Cys Glu Ala Asp Gly Ala Phe Cys Gly Ile Pro Ile
gtg aag aac tgg atg tgc tgc agt aac ttg tgt att ttt gcc tgc gta
Val Lys Asn Trp Met Cys Cys Ser Asn Leu Cys Ile Phe Ala Cys Val
ccc gag tat taagactgcc gtgatgtctt ctcctcccct c
Pro Glu Tyr

TABLE 4
DNA Sequence (SEQ ID NO:29) and Protein
Sequence (SEQ ID NO:30) of Tx6.12

a ttg gag aaa agg gat tgc cac gaa agg tgg gat tgg tgt cca gca tca
Leu Glu Lys Arg Asp Cys His Glu Arg Trp Asp Trp Cys Pro Ala Ser
atc ctt gga gtg ata tat tgc tgc gag gga tta att tgt ttt att gcc
Ile Leu Gly Val Ile Tyr Cys Cys Glu Gly Leu Ile Cys Phe Ile Ala
ttc tgc att tgatagtgat gtcttctcct cccctc
Phe Cys Ile

TABLE 5
DNA Sequence (SEQ ID NO:31) and
Protein Sequence (SEQ ID NO:32) of Tx6.13

a ttg gag aaa agg gat tgc caa gag aaa tgg gag ttt tgt ata gta ccg
Leu Glu Lys Arg Asp Cys Gln Glu Lys Trp Glu Phe Cys Ile Val Pro
atc ctt gga ttt gta tat tgc tgc cct ggc tta atc tgt ggc cct ttt
Ile Leu Gly Phe Val Tyr Cys Cys Pro Gly Leu Ile Cys Gly Pro Phe
gtc tgc gtt gat atc tgatgtcttc tcctcccatc
Val Cys Val Asp Ile

TABLE 6
DNA Sequence (SEQ ID NO:33) and
Protein Sequence (SEQ ID NO:34) of G28

ggatcc atg aaa ctg acg tgt gtg gtg atc gtt gtt gtg ctg ttg ttg
Met Lys Leu Thr Cys Val Val Ile Val Val Val Leu Leu Leu
aac gcc tgg aca ttc gtc tcc ata aat gga aag gcg aat cct ttt tgg
Asn Ala Trp Thr Phe Val Ser Ile Asn Gly Lys Ala Asn Pro Phe Trp
aag gca cgt gac gaa atg aag gac tcc gaa gtt tct gag ttg gag aaa
Lys Ala Arg Asp Glu Met Lys Asp Ser Glu Val Ser Glu Leu Glu Lys
agg agg aaa ccg acc tgc gtg tcg tat aac gtg ttt tgc gga gta ccg
Arg Arg Lys Pro Thr Cys Val Ser Tyr Asn Val Phe Cys Gly Val Pro
ctc gtt gga acc tac ctt tgc tgc agt ggc tta gtc tgt ctc gta gtc
Leu Val Gly Thr Tyr Leu Cys Cys Ser Gly Leu Val Cys Leu Val Val
tgc atc tagtactgat gtcttctact cccatctgtg ctacccctcg ag
Cys Ile

TABLE 7
DNA Sequence (SEQ ID NO:35) and
Protein Sequence (SEQ ID NO:36) of F763

ggatcc atg aaa ctg acg tgc gtg gtg atc gtt gct gtg ctg ttc ttg
Met Lys Leu Thr Cys Val Val Ile Val Ala Val Leu Phe Leu
acc gcc tgg aca ttc gtc acg gct gat gac tcc ata aat ggg ttg gag
Thr Ala Trp Thr Phe Val Thr Ala Asp Asp Ser Ile Asn Gly Leu Glu
aat ctt ttt ccg aag gca cgt cac gaa atg agg aaa ccc gaa gcc tct
Asn Leu Phe Pro Lys Ala Arg His Glu Met Arg Lys Pro Glu Ala Ser
aga tcg aga ggg agg tgc cgt cct cgt ggt atg ttc tgt ggc ttt ccg
Arg Ser Arg Gly Arg Cys Arg Pro Arg Gly Met Phe Cys Gly Phe Pro
aaa cct gga cca tac tgc tgc aat ggc tgg tgc ttt ttc gtc tgc atc
Lys Pro Gly Pro Tyr Cys Cys Asn Gly Trp Cys Phe Phe Val Cys Ile
taaaactgcc gtgatgtgtt ctactcccat ctgtgctacc cctcgag

TABLE 8
DNA Sequence (SEQ ID NO:37) and Protein
Sequence (SEQ ID NO:38) of F080

ggatcc atg aaa ctg acg tgc gtg gtg gtc gtt gct gtg ctg ttc ttg
Met Lys Leu Thr Cys Val Val Val Val Ala Val Leu Phe Leu
aac gcc tgg aca ttc gcc acg gct gtt gac tcc aaa cat gca ctg gcg
Asn Ala Trp Thr Phe Ala Thr Ala Val Asp Ser Lys His Ala Leu Ala
aaa ctt ttt atg aag gca cgt gac gaa atg tat aac ccc gat gcc act
Lys Leu Phe Met Lys Ala Arg Asp Glu Met Tyr Asn Pro Asp Ala Thr
aaa ttg gac gat aag aga tgg tgc gct tta gat ggt gaa ctt tgt atc
Lys Leu Asp Asp Lys Arg Trp Cys Ala Leu Asp Gly Glu Leu Cys Ile
ata ccg gtc att ggg tcc ata ttt tgc tgc cat ggc ata tgt atg atc
Ile Pro Val Ile Gly Ser Ile Phe Cys Cys His Gly Ile Cys Met Ile
tac tgc gtc tagttgaact gccgtgatgt cttctactcc cctctgtgct
Tyr Cys Val
acccctggtt tgatctttga ttgccctgtg cccttcactg attatgaatc cctctgatcc
tactctctga agacctcttg gggtccaaca tccaaataaa gcgacatccc aaaaaaaaaa
aaaaaaaaaa

TABLE 9
DNA Sequence (SEQ ID NO:39) and Protein
Sequence (SEQ ID NO:40) of F008

ggatcc atg aaa ctg acg tgt gtg gtg atc gtt gct gtg ctg ttc ttg
Met Lys Leu Thr Cys Val Val Ile Val Ala Val Leu Phe Leu
acc gcc tgg aca ttc gtc acg gct gac tcc ata cgt gca ctg gag gat
Thr Ala Trp Thr Phe Val Thr Ala Asp Ser Ile Arg Ala Leu Glu Asp
ttt ttt gcg aag gca cgt gac gaa atg gaa aac agc gga gct tct cca
Phe Phe Ala Lys Ala Arg Asp Glu Met Glu Asn Ser Gly Ala Ser Pro
ttg aac gag aga gac tgc cga cct gta ggt caa tat tgt ggc ata ccg
Leu Asn Glu Arg Asp Cys Arg Pro Val Gly Gln Tyr Cys Gly Ile Pro
tat aag cac aac tgg cga tgc tgc agt cag ctt tgt gca att atc tgt
Tyr Lys His Asn Trp Arg Cys Cys Ser Gln Leu Cys Ala Ile Ile Cys
gtt tcc taacccctct gatcctactc tctgaagacc tccgggattc aacatccaaa
Val Ser
taaagcgaca tcccgatnaa aaaaaangaa aaaaaaaaaa aaaa

TABLE 10
DNA Sequence (SEQ ID NO:41) and
Protein Sequence (SEQ ID NO:42) of G18

ggatcc atg aaa ctg acg tgt gtg gtg atc gtt gct gtg cta ttc ttg
Met Lys Leu Thr Cys Val Val Ile Val Ala Val Leu Phe Leu
acc gcc tgg aca ttc gtc acg gct gat gac acc aga tat aaa ctg gag
Thr Ala Trp Thr Phe Val Thr Ala Asp Asp Thr Arg Tyr Lys Leu Glu
aat cct ttt ctg aag gca cgc aac gaa ctg cag aaa cac gaa gcc tct
Asn Pro Phe Leu Lys Ala Arg Asn Glu Leu Gln Lys His Glu Ala Ser
caa ctg aac gag aga ggc tgc ctt gac cca ggt tac ttc tgt ggg acg
Gln Leu Asn Glu Arg Gly Cys Leu Asp Pro Gly Tyr Phe Cys Gly Thr
ccg ttt ctt gga gca tac tgc tgc ggt ggc att tgc ctt att gtc tgc
Pro Phe Leu Gly Ala Tyr Cys Cys Gly Gly Ile Cys Leu Ile Val Cys
ata gaa acg taaaggcttg atgtcttcta ctcccatctg tgctacccct cgag
Ile Glu Thr

TABLE 11
DNA and Amino Acid Sequences of Mu—O Conopeptides

| | |
|---|---|
| Name: Ca6.1 | |
| Species: caracteristicus | |
| Isolated: No | |
| Cloned: Yes | |
| DNA Sequence: | |
| ATGAAACTGACGTGCGTGATGATCGTTGCTGTGCTGTTCTTGACCGCCTGGACATTCGT | (SEQ ID NO:43) |
| CACGGCTGATGACTCCATTAATGCACTGGAGGATCTTTTTTCGAAGGCACGTGACGAAA | |
| TGGAAAACGGCGAAGCTTCTACATTGAACGAGAGAGACTGCGAAGCAGATGGTGCATT | |
| TTGTGGTATCCCAATTGTGAAGAACTGGATGTGCTGCAGTAACTTGTGTATTTTTGCCTG | |
| CGTACCCGAGTATTAAGACTGCCGTGATGTCTTCTCCTCCCCTC | |
| Translation: | |
| MKLTCVMIVAVLFLTAWTFVTADDSINALEDLFSKARDEMENGEASTLNERDCEADGAFC | (SEQ ID NO:44) |
| GIPIVKNWMCCSNLCIFACVPEY | |
| Toxin Sequence: | |
| Asp-Cys-Xaal-Ala-Asp-Gly-Ala-Phe-Cys-GlY-Ile-Xaa3-Ile-Val-Lys-Asn-Xaa4-Met-Cys-Cys-Ser- | (SEQ ID NO:45) |
| Asn-Leu-Cys-Ile-Phe-Ala-Cys-Val-Xaa3-Xaa1-Xaa5-\hat{ } | |
| Name: Nb6.1 | |
| Species: nobilis | |
| Isolated: No | |
| Cloned: Yes | |
| DNA Sequence: | |
| ATGAAACTGACGTGTGTGGTGATCGTTGCTGTGCTGTTCTTGACCGCCTGGACATTCGT | (SEQ ID NO:46) |
| CATGGCTGATGACCCCAGAGATGGAGCGGAGATTAGAAGCATGGTAAGGGGGGAACCT | |
| CTGTCGAAGGCACGTGACGAAATGAACCCCGAAGCCTCTAAATTGGAGAAAAGGGCGT | |
| GCCGCCAAAAATACGAATTTTGTCTAGTACCGATCATTGGATACATATATTGCTGCGCT | |
| GGCTTAATCTGTGGTCCTTTCGTCTGCCTTTGATAGTGATGTCTTCTACTGCCATCTGTG | |
| CTACCCCTGGCTTGATCTTTGATAGGCGTTGTTGCCCTTCACTGGTTTATGAACCCTCTG | |
| ATCATACTCTCTGGACCCTTGGGGGTCCAACATCCAAATAAAGCGACATCCCAAAAAA | |
| AAAAAAAAAAAA | |
| Translation: | |
| MKLTCVVIVAVLFLTAWTFVMADDPRDGAEIRSMVRGEPLSKARDEMNPEASKLEKRACR | (SEQ ID NO:47) |
| QKYEFCLVPIIGYIYCCAGLICGPFVCL | |
| Toxin Sequence: | |
| Ala-Cys-Arg-Gln-Lys-Xaa5-Xaa1-Phe-Cys-Leu-Val-Xaa3-Ile-Ile-Gly-Xaa5-Ile-Xaa5-Cys-Cys- | (SEQ ID NO:48) |
| Ala-Gly-Leu-Ile-Cys-Gly-Xaa3-Phe-Val-Cys-Leu-\hat{ } | |
| Name: Tx6.12 | |
| Species: textile | |
| Isolated: No | |
| Cloned: Yes | |
| DNA Sequence: | |
| ATTGGAGAAAAGGGATTGCCACGAAAGGTGGGATTGGTGTCCAGCATCAATCCTTGGA | (SEQ ID NO:49) |
| GTGATATATTGCTGCGAGGGATTAATTTGTTTTATTGCCTTCTGCATTTGATAGTGATGT | |
| CTTCTCCTCCCCTC | |
| Translation: | |
| LEKRDCHERWDWCPASILGVIYCCEGLICFIAFCI | (SEQ ID NO:50) |
| Toxin Sequence: | |
| Asp-Cys-His-Xaa1-Arg-Xaa4-Asp-Xaa4-Cys-Xaa3-Ala-Ser-Ile-Leu-Gly-Val-Ile-Xaa5-Cys-Cys- | (SEQ ID NO:51) |
| Xaa1-Gly-Leu-Ile-Cys-Phe-Ile-Ala-Phe-Cys-Ile-\hat{ } | |
| Name: Tx6.13 | |
| Species: textile | |
| Isolated: No | |
| Cloned: Yes | |
| DNA Sequence: | |
| ATTGGAGAAAAGGGATTGCCAAGAGAAATGGGAGTTTTGTATAGTACCGATCCTTGGA | (SEQ ID NO:52) |
| TTTGTATATTGCTGCCCTGGCTTAATCTGTGGCCCTTTTGTCTGCGTTGATATCTGATGTC | |
| TTCTCCTCCCATC | |
| Translation: | |
| LEKRDCQEKWEFCIVPILGFVYCCPGLICGPFVCVDI | (SEQ ID NO :53) |
| Toxin Sequence: | |
| Asp-Cys-Gln-Xaa1-Lys-Xaa4-Xaa1-Phe-Cys-Ile-Val-Xaa3-Ile-Leu-Gly-Phe-Val-Xaa5-Cys-Cys- | (SEQ ID NO:54) |
| Xaa3-Gly-Leu-Ile-Cys-Gly-Xaa3-Phe-Val-Cys-Val-Asp-Ile-\hat{ } | |
| Name: Ts6.1 | |
| Species: tessulatus | |
| Isolated: No | |
| Cloned: Yes | |
| DNA Sequence: | |
| GGATCCATGAAACTGACGTGCATGGTGATCGTTGTTGTGCTGTTGTTGAACGCCTGGAC | (SEQ ID NO:55) |
| ATTCGTCTCCATAAATGGAAAGGCGAATCGTTTTTGGAAGGCACGTGACGAAATGAAG | |
| GACTCCGAAGTTTCTGAATTGGAGAAAAGGAGGAAACCGACCTGCCTGAAGCAGGACA | |
| AGTTTTGCATAATACCGCTCATTGGAACCCTTTATTGCTGCAGTGGGTTAATCTGTGGGT | |
| TTTTTGTCTGCGTCCCAAAGCCGTTCTGATGTCTTCTACTGCCATCTGTGCTACCCTGG | |
| CTTGATCTTTGATTGGCGTGTGCCCTTCACTGGTTATGAACCCCTCTGATCCTACTGTCT | |
| GGACGCCTCGGGCGTCCAACGTCCAAATAAAGCGACATCCCAATAAAAAAAAAAAAA | |
| AAAAAA | |
| Translation: | |
| MKLTCMVIVVVLLLNAWTFVSINGKANRFWKARDEMKDSEVSELEKRRKPTCLKQDKFCI | (SEQ ID NO:56) |
| IPLIGTLYCCSGLICGFFVCVPKPF | |
| Toxin Sequence: | |
| Xaa3-Thr-Cys-Leu-Lys-Gln-Asp-Lys-Phe-Cys-Ile-Ile-Xaa3-Leu-Ile-Gly-Thr-Leu-Xaa5-Cys-Cys- | (SEQ ID NO:57) |
| Ser-Gly-Leu-Ile-Cys-Gly-Phe-Phe-Val-Cys-Val-Xaa3-Lys-Xaa3-Phe-\hat{ } | |
| Name: Ts6.5 | |
| Species: tessulatus | |
| Isolated: No | |
| Cloned: Yes | |
| DNA Sequence: | |
| GGATCCATGAAACTGACGTGTGTGGTGATCGTTGTTGTGCTGTTGTTGAACGCCTGGAC | (SEQ ID NO:58) |
| ATTCGTCTCCATAAATGGAAAGGCGAATCCTTTTTGGAAGGCACGTGACGAAATGAAG | |
| GACTCCGAAGTTTCTGAGTTGGAGAAAAGGAGGAAACCGACCTGCGTGTCGTATAACG | |
| TGTTTTGCGGAGTACCGCTCGTTGGAACCTACCTTTGCTGCAGTGGCTTAGTCTGTCTCG | |
| TAGTCTGCATCTAGTACTGATGTCTTCTACTCCCATCTGTGCTACCCCTCGAG | |
| Translation: | |
| MKLTCVVIVVVLLLNAWTFVSINGKANPFWKARDEMKDSEVSELEKRRKPTCVSYNVFCG | (SEQ ID NO:59) |
| VPLVGTYLCCSGLVCLVVCI | |
| Toxin Sequence: | |
| Xaa3-Thr-Cys-Val-Ser-Xaa5-Asn-Val-Phe-Cys-Gly-Val-Xaa3-Leu-Val-Gly-Thr-Xaa5-Leu-Cys- | (SEQ ID NO:60) |
| Cys-Ser-Gly-Leu-Val-Cys-Leu-Val-Val-Cys-Ile-\hat{ } | |
| Name: Ca6.2 | |
| Species: caracteristicus | |
| Isolated: No | |
| Cloned: Yes | |
| DNA Sequence: | |
| GGATCCATGAAACTGACGTGCATGGTGATCGTTGCTGTGCTGTTCTTGACCGCCTGGAC | (SEQ ID NO:61) |
| ATTCGTCACGGCTGATGACTCCATTAATGCACTGGAGGATCTTTTTTCGAAGGCACGCG | |
| ACGAAATGGAAAACGGCGAAGCTTCTACATTGAACGAGAGAGACTGCGAAGCAGATG | |
| GTGCATTTTGTGGTATCCCAACTGTGAAGAACTGGATGTGCTGCAGTAACTTGTGTATTT | |
| TTGCCTGCGTACCCGAGTATTAAGACTGCCGTGATGTCTTCTGCTCCCCTCTGTGCTACC | |
| TGGCTTGATCTTTGATTGGCGCGTGCCCTTCACTGGTTATGAACCCCTCTGATCCTACTC | |
| TC | |
| Translation: | |
| MKLTCMVIVAVLFLTAWTFVTADDSINALEDLFSKARDEMENGEASTLNERDCEADGAFC | (SEQ ID NO:62) |
| GIPTVKNWMCCSNLCIFACVPEY | |
| Toxin Sequence: | |
| Asp-Cys-Xaa1-Ala-Asp-Gly-Ala-Phe-Cys-Gly-Ile-Xaa3-Thr-Val-Lys-Asn-Xaa4-Met-Cys-Cys-Ser- | (SEQ ID NO:63) |
| Asn-Leu-Cys-Ile-Phe-Ala-Cys-Val-Xaa3-Xaa1-Xaa5-\hat{ } | |
| Name: Ep6.3 | |
| Species: episcopatus | |
| Isolated: No | |
| Cloned: Yes | |
| DNA Sequence: | |
| GGATCCATGAAACTGACGTGTGTGGTGATCATTGCTGTGCTGTTCTTGACCGCCTGGAC | (SEQ ID NO:64) |
| ATTCGTCATGGCTGATGACTCCAACAATGGACTGGCGAATCATTTTTCGAAATCACGTG | |
| ACGAAATGGAGGACCCCGAAGCTTCTAAATTGGAGAAAAGAAGGGATTGCCAAGAAG | |
| AATGGGAATTTTGTATAGTACCGATCCTTGGATTCGTATATTGCTGCCCTGGCTTAATCT | |
| GTGGTCCTTTCGTCTGCGTTTAATACTGATGTCTTCTACTCCCATCTGTGCTACACCTGG | |
| CTTGATCTTTGATAGGCGTGTGCCCTTCACTGGTTATAAACCCCTCTGATCCTACTCTCT | |
| GGACGCCTCGGGGGCCCAACATCCAAATAAAGCAACATCCCAATGAANAAAAAA | |
| Translation: | |
| MKLTCVVIIAVLFTAWTFVMADDSNNGLANHFSKSRDEMEDPEASKLEKRRDCQEEWEF | (SEQ ID NO:65) |
| CIVPILGFVYCCPGLICGPFVCV | |
| Toxin Sequence: | |
| Asp-Cys-Gln-Xaa1-Xaa1-Xaa4-Xaa1-Phe-Cys-Ile-Val-Xaa3-Ile-Leu-Gly-Phe-Val-Xaa5-Cys-Cys- | (SEQ ID NO:66) |
| Xaa3-Gly-Leu-Ile-Cys-Gly-Xaa3-Phe-Val-Cys-Val-\hat{ } | |

Xaa1 = Glu or γ-Carboxy Glu
Xaa2 = Gln or pyroglu
Xaa3 = Pro or Hydroxy Pro
Xaa4 = Trp or Bromo Trp
Xaa5 = Tyr, ¹²⁵I-Tyr, Mono-Iodo Tyr, Di-Iodo Tyr, O-sulpho-Tyr or O-Phospho-Tyr
\hat{ } = Free-carboxyl C-term or Amidated C-term, preferably Free-carboxyl
# = Free-carboxyl C-term or Amidated C-term, preferably Amidated.

### Example 2

### Effect of Intrathecal Administration of MrV,IB

Male C57 black mice (20-25g) were obtained from Charles River Laboratories. These mice and the animals used in the other examples were housed in a temperature controlled (23°±3° C.) room with a 12 hour light-dark cycle with free access to food and water. All animals were euthanized in accordance with Public Health Service policies on the humane care of laboratory animals.

Intrathecal (it) drug injections were performed as described (Hylden and Wilcox, 1980). MrVIB (10 nmol) or vehicle was administered in a volume of 5 μl. Duration of hind-limb paralysis was assessed. This experiment revealed that injection of 10 nmols of MrVIB into the intrathecal space of C57 black mice produced a long-lasting paralysis (>20 hrs) of the animal. The injection initially produced a paralysis of the hind-limbs, but over the following 30 minutes resolved into paralysis of the entire animal. Despite the long duration of anesthesia, the animals in this experiment recovered fully. Similar results were obtained with MrVIA. Similar results are also obtained with A657, F079, Ca6.1, Tx6.12, Tx6.13, G28, F763 and F080.

### Example 3

### Effect of MrVIB as a Local Anesthetic

Male Hartley guinea pigs (retired breeders) were obtained form Charles River Laboratories. The local anesthetic test was performed essentially as described (Bulbring and Wajda, 1945). On the day prior to test day, a patch on the back of the guinea pig was denuded of hair, first by shaving with electric clippers and subsequently with depilatory cream (Nair®). Depilatory cream was applied for five minutes and removed with a warm washcloth. The guinea pigs were dried and returned to their cages. On the following day, intradermal injections (0.1 ml vols) of lidocaine, bupivacaine, MrVIB or vehicle (0.5% cyclodextran) were made into the denuded patch. The injection produced a raised wheal on the surface of the skin which was circled with a felt-tipped pen. Typically, four injections were made on the back of each guinea pig. In some cases, guinea pigs were reused following at least one week of recovery and injecting into an unused portion of the skin.

The stimulus consisted of mild pin pricks (not hard enough to break the skin) with a 26G needle. The response is a localized skin twitch caused by contraction of cutaneous muscles. A unit test consisted of six uniform pin pricks, 3-5 seconds apart, within the injected area. Unit scores ranged from 0 (complete anesthesia) to 6 (no anesthesia). For potency experiments, the unit test was repeated at each site at five minute intervals for 30 minutes, and unit test scores summed (with 36 representing no anesthesia to 0 representing complete anesthesia. For duration experiments, unit tests were performed as described over the course of several hours to days.

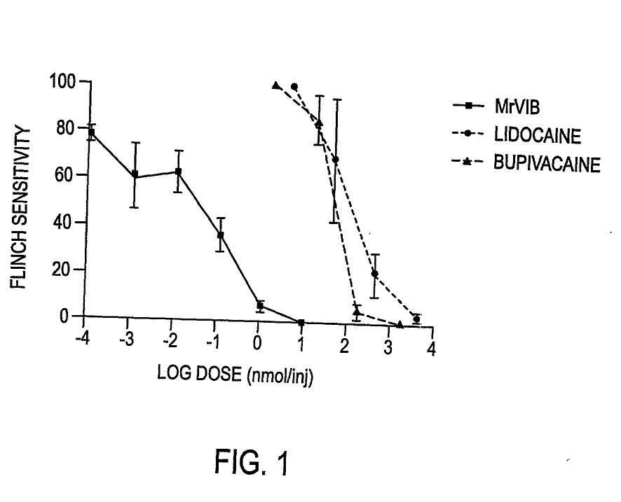

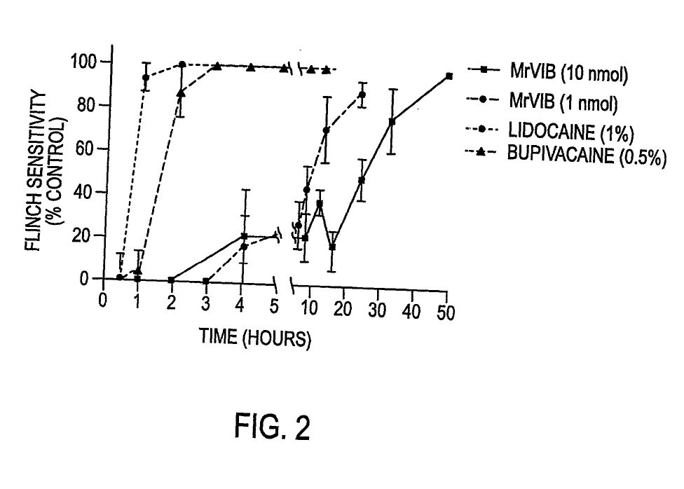

MrVIB produced a potent (FIG. 1) and long lasting (FIG. 2) local anesthetic effect in the intracutaneous wheat test in the guinea pig. The ED₅₀'s for this response (≈100 pmol) was at least two orders of magnitude greater than the ED₅₀'s for lidocaine and bupivacaine. Moreover, the duration of roughly equieffective doses of MrVIB (roughly 24 and 48 hours for full recovery following 1 and 10 nmol, respectively) was much longer than that of lidocaine and bupivacaine (≈30 and 90 minutes for fall recovery, respectively). As expected, bupivacaine had a slightly longer duration than lidocaine, consistent with clinical observations. It was seen during the experiment that the intracutaneous wheal consistently turned red several hours following injection of MrVIB, possibly suggesting an antigenic action. Similar results are obtained with MrVIA, A657, F079, Ca6.1, Tx6.12, Tx6.13, G28, F763 and F080.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### Bibliography

Barnay, G. et al. (2000). J. Med. Chem.

Bitan, G. et al. (1997). J. Peptide Res. 49:421-426.

Bodansky et al. (1966). Chem. Ind. 38:1597-98.

Bulbring, W. and Wajda, J. (1945). J. Pharmacol. Exp. Ther. 85:78-84.

Craik, D. J. et al. (1991). Toxicon 39:43-60.

Ettinger, L. J. et al. (1978). Cancer 41:1270-1273.

Fainzilber, M. et al. (1995). Biochemistry 34:8649-8656.

Hammerland et al. (1992). Eur. J. Pharmacol. 226:239-242.

Horiki, K. et al. (1978). Chemistry Letters 165-68.

Hubry, V. et al. (1994). Reactive Polymers 22:231-241.

Hylden, J. L. K. and Wilcox, G. (1980). Eur. J. Pharmacol. 67:313-316.

Kaiser et al. (1970). Anal. Biochem. 34:595.

Kapoor (1970). J. Pharm. Sci. 59:1-27.

Kornreich, W. D. et al. (1986). U.S. Pat. No. 4,569,967.

Kuzma, P. K. et al. (1997). Regional Anesthesia 22:543-551.

Luer, M. S. et al. (1993). Annals of Pharmacotherapy, 27:912-921.

McIntosh, J. M. et al. (1995). J. Biol Chem. 270:16796-16802.

McIntosh, J. M. et al. (1998). Methods Enzymol. 294:605-624.

Methoden der Organischen Chemie (Houben-Weyl): Synthese von Peptiden, E. Wunsch (Ed.), Georg Thieme Verlag, Stuttgart, Ger. (1974).

Nishiuchi, Y. et al. (1993). Int. J. Pept. Protein Res. 42:533-538.

Nowak, L. et al. (1984). Nature 307:462-465.

Olivera, B. M. et al. (1984). U.S. Pat. No. 4,447,356.

Olivera, B. M. et al. (1985). Science 230:1338-1343.

Olivera, B. M. et al. (1990). Science 249:257-263.

Ornstein, et al. (1993). Biorganic Medicinal Chemistry Letters 3:43-48.

Remington's Pharmaceutical Sciences, 18th Ed., Mack Publishing Co., Easton, Pa. (1990).

Rivier, J. R. et al. (1978). Biopolymers 17:1927-38.

Rivier, J. R. et al. (1987). Biochem. 26:8508-8512.

Rosenberg, G. J. et al. (1996). Clinics in Plastic Surgery 23:29.

Sambrook, J. et al. (1989). Molecular Cloning: A Laboratory Manual, 2nd Ed., Cold Spring Harbor Laboratory, Cold Spring Harbor, NY.

Schroder & Lubke (1965). The Peptides 1:72-75, Academic Press, NY.

Stewart and Young, Solid-Phase Peptide Synthesis, Freeman & Co., San Francisco, Calif. (1969).

Terlau et al. (1996). J. Neurophysiol. 76:1423-1429.

Vale et al. (1978). U.S. Pat. No. 4,105,603.

Van de Steen, P. et al. (1998). Critical Rev. in Biochem. and Mol. Biol. 33:151-208.

Zhou L. M., et al. (1996). J. Neurochem. 66:620-628.

Zimm, S. et al. (1984). Cancer Research, 44:1698-1701.

U.S. Pat. No. 3,842,067.

U.S. Pat. No. 3,862,925.

U.S. Pat. No. 3,972,859.

U.S. Pat. No. 4,352,883

U.S. Pat. No. 4,353,888

U.S. Pat. No. 4,883,666

U.S. Pat. No. 4,968,733

U.S. Pat. No. 4,976,859

U.S. Pat. No. 5,082,670

U.S. Pat. No. 5,084,350

U.S. Pat. No. 5,158,884

U.S. Pat. No. 5,284,761

U.S. Pat. No. 5,364,769

U.S. Pat. No. 5,514,774.

U.S. Pat. No. 5,531,001.

U.S. Pat. No. 5,534,615

U.S. Pat. No. 5,545,723

U.S. Pat. No. 5,550,050.

U.S. Pat. No. 5,591,821.

U.S. Pat. No. 5,618,531

U.S. Pat. No. 5,719,264

U.S. Pat. No. 5,844,077.

U.S. Pat. No. 5,859,186.

PCT Published Application WO 92/19195.

PCT Published Application WO 94/25503.

PCT Published Application WO 95/01203.

PCT Published Application WO 95/05452.

PCT Published Application WO 96/02286.

PCT Published Application WO 96/02646.

PCT Published Application WO 96/11698.

PCT Published Application WO 96/40871.

PCT Published Application WO 96/40959.

PCT Published Application WO 97/12635.

PCT Published Application WO 98/03189.

PCT Published Application WO 00/23092.

What is claimed is:

1. A method of alleviating pain which comprises administering to a mammal that is either exhibiting pain or is about to be subjected to a pain-causing event a pain-alleviating amount of an active agent comprising a μO-conopeptide, derivative or pharmaceutically acceptable salt or solvate thereof.

2. The method of claim 1, wherein said μO-conopeptide has the general formula I:
Xaa₁-Xaa₂-Cys-Xaa₃-Xaa₄-Xaa₅-Xaa₆-Xaa₇-Xaa₈-Cys-Xaa₉-Xaa₁₀-Xaa₁₁-Xaa₁₂-Xaa₁₃-Xaa₁₄-Xaa₁₅-Xaa₁₆-Xaa₁₇-Cys-Cys-Xaa₁₈-Xaa₁₉-Xaa₂₀-Xaa₂₁-(ys-Xaa₂₂-Xaa₂₃-Xaa₂₄-Xaa₂₅-Cys-Xaa₂₆-Xaa₂₇-Xaa₂₈-Xaa₂₉-Xaa₃₀ (SEQ ID NO:1),
wherein Xaa₁ is des-Xaa₁, Pro, hydroxy-Pro (Hyp), Arg, Lys, ornithine, homo-Lys, homoarginine, nor-Lys, N-methyl-Lys, N,N′-dimethyl-Lys, N,N′,N″-trimethyl-Lys or any synthetic basic amino acid; Xaa₂ is des-Xaa₂, Ala, Gly, Asp, Glu, γ-carboxy-glutamate (Gla), any synthetic acidic amino acid, Thr, Ser, g-Thr (where g is glycosylation), g-Ser, Trp (D or L), neo-Trp or halo-Trp (D or L) or Xaa₂ may be pyroglutamate if Xaa₁ is des-Xaa₁; Xaa₃ is Arg, Lys, ornithine, homo-Lys, homoarginine, nor-Lys, N-methyl-Lys, N,N′-dimethyl-Lys, N,N′,N″-trimethyl-Lys, any synthetic basic amino acid, Ser, Thr, g-Ser, g-Thr, Ala, an aliphatic amino acid bearing linear or branched saturated hydrocarbon chains such as Leu (D or L), Ile and Val or non-natural derivatives of the aliphatic amino acid, His, Glu, Gln, Gla, Asp, Asn or any synthetic acidic amino acid; Xaa₄ is Glu, Gla, Gln, Asp, Asn, any synthetic acidic amino acid, Lys, Arg, ornithine, homo-Lys, homoarginine, nor-Lys, N-methyl-Lys, N,N′-dimethyl-Lys, N,N′,N″-trimethyl-Lys, any synthetic basic amino acid, Ala, an aliphatic amino acid bearing linear or branched saturated hydrocarbon chains such as Leu (D or L), Ile and Val or non-natural derivatives of the aliphatic amino acid, Ser, Thr, Pro, Hyp, g-Ser, g-Thr, g-Hyp or any synthetic hydroxylated amino acid; Xaa₅ is Lys, Arg, ornithine, homo-Lys, homoarginine, nor-Lys, N-methyl-Lys, N,N′-dimethyl-Lys, N,N′,N″-trimethyl-Lys, any synthetic basic amino acid, Tyr, meta-Tyr, ortho-Tyr, nor-Tyr, mono-halo-Tyr, di-halo-Tyr, O-sulpho-Tyr, O-phospho-Tyr, nitro-Tyr, an aliphatic amino acid bearing linear or branched saturated hydrocarbon chains such as Leu (D or L), Ile and Val or non-natural derivatives of the aliphatic amino acid, Glu, Gla, Gln, Asp, Asn, any synthetic acidic amino acid, Pro or Hyp; Xaa₆ is Trp (D or L), neo-Trp, halo-Trp (D or L), Gly, Tyr, meta-Tyr, ortho-Tyr, nor-Tyr, mono-halo-Tyr, di-halo-Tyr, O-sulpho-Tyr, O-phospho-Tyr, nitro-Tyr, Glu, Gla, Gln, Asp, Asn, any synthetic acidic amino acid; Xaa₇ is Glu, Gla, Gln, Asp, Asn, any synthetic acidic amino acid, Met, norleucine (Nle), Ala, an aliphatic amino acid bearing linear or branched saturated hydrocarbon chains such as Leu (D or L), Ile and Val or non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr, Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa₈ is Leu, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₉ is Pro, Hyp, Gly, an aliphatic amino acid bearing linear or branched saturated hydrocarbon chains such as Leu (D or L), Ile and Val or non-natural derivatives of the aliphatic amino acid; Xaa₁₀ is Thr, Ser, g-Thr, g-Ser, Ala, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₁₁, is Pro, Hyp, Ser, Thr, g-Hyp, g-Ser, g-Thr or any hydroxylated amino acid; Xaa₁₂ is an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa₁₃ is Pro, Hyp, an aliphatic amino acid bearing linear or branched saturated hydrocarbon chains such as Leu (D or L), Ile and Val or non-natural derivatives of the aliphatic amino acid, Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa₁₄ is Gly, His, Lys, Arg, ornithine, homo-Lys, homoarginine, nor-Lys, N-methyl-Lys, N,N-dimethyl-Lys, N,N′,N″-trimethyl-Lys or any synthetic basic amino acid; Xaa₁₅ is des-Xaa₁₅, Ser, Thr, g-Ser, g-Thr, Val, Asn, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid;Xaa₁₆ is Met, Nle, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L), any synthetic aromatic amino acid, Arg, Lys, ornithine, homo-Lys, homoarginine, nor-Lys, N-methyl-Lys, N,N′-dimethyl-Lys, N,N′,N″-trimethyl-Lys or any synthetic basic amino acid; Xaa₁₇ is Pro, Hyp, Ser, Thr, g-Hyp, g-Ser, g-Thr, any hydroxylated amino acid, Ala, Met, Leu, Glu, Gla, Gln, Asp, Asn, any synthetic acidic amino acid, Tyr, meta-Tyr, ortho-Tyr, nor-Tyr, mono-halo-Tyr, di-halo-Tyr, O-sulpho-Tyr, O-phospho-Tyr, nitro-Tyr, His or Gly; Xaa₁₈ is Gly, Ala, Ser, Thr, Pro, Hyp, g-Ser, g-Thr, g-Hyp, Glu, Asn or Gln; Xaa₁₉ is Leu, Gly, Asn, Trp (D or L), neo-Trp or halo-Tip (D or L); Xaa₂₀ is des-Xaa₂₀, Leu or Trp (D or L), neo-Trp or halo-Tip (D or L); Xaa₂₁ is des-Xaa₂₁ or an aliphatic amino acid bearing linear or branched saturated hydrocarbon chains such as Leu (D or L), Ile and Val or non-natural derivatives of the aliphatic amino acid; Xaa₂₂ is des-Xaa₂₂, Gly, Met, Nle, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₂₃ is des-Xaa₂₃, Pro, Hyp, Ala, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Tip (D or L), neo-Trp, halo-Trp (D or L) or any synthetic aromatic amino acid; Xaa₂₄ is Ala, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₂₅ is Ala, an aliphatic amino acid bearing linear or branched saturated hydrocarbon chains such as Leu (D or L), Ile and Val or non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr or Phe; Xaa₂₆ is an aliphatic amino acid bearing linear or branched saturated hydrocarbon chains such as Leu (D or L), Ile and Val or non-natural derivatives of the aliphatic amino acid; Xaa₂₇ is des-Xaa₂₇, Asp, Glu, Gla, Pro, Hyp, Ser, Thr, g-Hyp, g-Ser, g-Ser or any synthetic hydroxylated amino acid; Xaa₂₈ is des-Xaa₂₈, Glu, Gla, Gln, Asp, Asn, any synthetic acidic amino acid, Lys, Arg, ornithine, homo-Lys, homoarginine, nor-Lys, N-methyl-Lys, N,N′-dimethyl-Lys, N,N′,N′″-trimethyl-Lys, any synthetic basic amino acid, Ile, Ser, Thr, g-Ser or g-Thr; Xaa₂₉ is des-Xa₉, Pro, Hyp, Tyr, meta-Tyr, ortho-Tyr, nor-Tyr, mono-halo-Tyr, di-halo-Tyr, O-sulpho-Tyr, O-phospho-Tyr or nitro-Tyr; Xaa₃₀ is des-Xaa₃₀ or Phe.

3. The method of claim 2, wherein said μO-conopeptide is selected from the group consisting of:
Ala-Cys-Arg-Gln-Xaa₁-Xaa₂-Xaa₃-Phe-Cys-Leu-Val-Xaa₄-Ile-Ile-Gly-Xaa₂-Ile-Xaa₂-Cys-Cys-Ala-Gly-Leu-Ile-Cys-Gly-Xaa₄-Phe-Val-Cys-Leu (SEQ ID NO:3);
Xaa₄-Thr-Cys-Leu-Xaa₁-Gln-Asp -Xaa₁-Phe-Cys-Ile-Ile-Xaa₄-Leu-Ile-Gly-Thr-Leu-Xaa₂-Cys-Cys-Ser-Gly-Leu-Ile-Cys-Gly-Phe-Phe-Val-Cys-Val-Xaa₄-Xaa₁-Xaa₄-Phe (SEQ ID NO:4);
Asp-Cys-Xaa₃-Ala-Asp-Gly-Ala-Phe-Cys-Gly-Ile-Xaa₄-Ile-Vla-Xaa₁-Asn-Xaa₅-Met-Cys-Cys-Ser-Asn-Leu-Cys-Ile-Phe-Ala-Cys-Val-Xaa₄-Xaa₃-Xaa₂ (SEQ ID NO:5);
Asp-Cys-His-Xaa₃-Arg-Xaa₅-Asp-Xaa₅-Cys-Xaa₄-Ala-Ser-Ile-Leu-Gly-Val-Ile-Xaa₂-Cys-Cys-Xaa₃-Gly-Leu-Ile-Cys-Phe-Ile-Ala-Phe-Cys-Ile (SEQ ID NO:6);
Asp-Cys-Gln-Xaa₃-Xaa₁-Xaa₅-Xaa₃-Phe-Cys-Ile-Val-Xaa₄-Ile-Leu-Gly-Phe-Val-Xaa₂-Cys-Cys-Xaa₄-Gly-Leu-Ile-Cys-Gly-Xaa₄-Phe-Val-Cys-Val-Asp-Ile (SEQ ID NO:7);
Xaa₄-Thr-Cys-Val-Ser-Xaa₂-Asn-Val-Phe-Cys-Gly-V al-Xaa₄-Leu-Val-Gly-Thr-Xaa₂-Leu-Cys-Cys-Ser-Gly-Leu-Val-Cys-Leu-Val-Val-Cys-Ile (SEQ ID NO:8);
Cys-Arg-Xaa₄-Arg-Gly-Met-Phe-Cys-Gly-Phe-Xaa₄-Xaa₁-Xaa₄-Gly-Xaa₄-Xaa₂-Cys-Cys-Asn-Gly-Xaa₅-Cys-Phe-Phe-Val-Cys-Ile (SEQ ID NO:9);
Arg-Xaa₅-Cys-Ala-Leu-Asp-Gly-Xaa₃-Leu-Cys-Ile-Ile-Xaa₄-Val-Ile-Gly-Ser-Ile-Phe-Cys-Cys-His-Gly-Ile-Cys-Met-Ile-Xaa₂-Cys-Val (SEQ ID NO:10);
Asp-Cys-Arg-Xaa₄-Val-Gly-Gln-Xaa₂-Cys-Gly-Ile-Xa₄-Xaa₂-Xaa₁-His-Asn-Xaa₅-Arg-Cys-Cys-Ser-Gln-Leu-Cys-Ala-Ile-Ile-Cys-Val-Ser (SEQ ID NO:11);
Gly-Cys-Leu-Asp-Xaa₄-Gly-Xaa₂-Phe-Cys-Gly-Thr-Xaa₄-Phe-Leu-Gly-Ala-Xaa₂-Cys-Cys-Gly-Gly-Ile-Cys-Leu-Ile-Val-Cys-Ile-Xaa₃-Thr (SEQ ID NO:12);
Asp-Cys-Xaa₃-Ala-Asp-Gly-Ala-Phe-Cys-Gly-Ile-Xaa₄-Thr-Val-Xaa₁-Asn-Xaa₅-Met-Cys-Cys-Ser-Asn-Leu-Cys-Ile-Phe-Ala-Cys-Val-Xaa₄-Xaa₃-Xaa₂ (SEQ ID NO:14);
Asp-Cys-Gln-Xaa₃-Xaa₃-Xaa₅-Xaa₃-Phe-Cys-Ile-Val-Xaa₄-Ile-Leu-Gly-Phe-Val-Xaa₂-Cys-Cys-Xaa₄-Gly-Leu-Ile-Cys-Gly-Xaa₄-Phe-Val-Cys-Val (SEQ ID NO:15);
Ala-Cys-Arg-Gln-Xaa₁-Xaa₂-Xaa₃-Phe-Cys-Leu-Val-Xaa₄-Ile-Ile-Gly-Xaa₂-Ile-Xaa₂-Cys-Cys-Ala-Gly-Leu-Ile-Cys-Gly-Xaa₄-Phe-Val-Cys-Leu (SEQ ID NO:16); and
Xaa₄-Thr-Cys-Leu-Xaa₁-Gln-Asp-Xaa₁-Phe-Cys-Ile-Ile-Xaa₄-Leu-Ile-Gly-Thr-Leu-Xaa₂-Cys-Cys-Ser-Gly-Leu-Ile-Cys-Gly-Phe-Phe-Val-Cys-Val-Xaa₄-Xaa₁-Xaa₄-Phe (SEQ ID NO:17),
wherein Xaa₁ is Lys, N-methy-Lys, N,N-dimethyl-Lys or N,N,N-trimethyl-Lys; Xaa₂ is Tyr, mono-halo-Tyr, di-halo-Tyr, O-sulpho-Tyr, O-phospho-Tyr or nitro-Tyr; Xaa₃ is Glu or gamma-carboxy-Glu (Gla); Xaa₄ is Pro or hydroxy-Pro; Xaa₅ is Trp or halo-Trp; and the C-terminus contains a carboxyl or amide group.

4. The method of claim 1, wherein the pain is chronic pain or acute inflammatory pain.

5. The method of claim 1, wherein the pain is neuropathic pain.

6. The method of claim 1, wherein the active agent is administered prior to surgery.

7. The method of claim 1, wherein the active agent is administered as a spinal anesthetic.

8. The method of claim 1, wherein the active agent is administered as a local anesthetic.

9. The method of claim 8, wherein the active agent is administered topically.

10. The method of claim 9, wherein the pain is pain in epithelial tissue.

11. The method of claim 10, wherein the pain in epithelial tissue is associated with damage or loss to the epithelial tissue.

12. The method of claim 9, wherein the pain is in the cornea.

13. The method of claim 1, wherein said active agent is administered in an amount from about 1 ng to about 1000 mg per day.

14. The method of claim 1, wherein said active agent is administered in an amount from about 100 ng to about 100 mg per day.

15. The method of claim 1, wherein said active agent is administered in an amount from about 1 μg to about 10 mg per day.

16. An isolated nucleic acid comprising a nucleic acid coding for a μO-conopeptide precursor comprising an amino acid sequence selected from the group of amino acid sequences set forth in Tables 1-11.

17. The nucleic acid of claim 16 wherein the nucleic acid comprises a nucleotide sequence selected from the group of nucleotide sequences set forth in Tables 1-11 or their complements.

18. A substantially pure μO-conopeptide precursor comprising an amino acid sequence selected from the group of amino acid sequences set forth in Tables 1-11.

19. A substantially pure μO-conotopeptide having the generic formula I:
Xaa₁-Xaa₂-Cys-Xaa₃-Xaa₄-Xaa₅-Xaa₆-Xaa₇-Xaa₈-Cys-Xaa₉-Xaa₁₀-Xaa₁₁-Xaa₁₂-Xaa₁₃-Xaa₁₄-Xaa₁₅-Xaa₁₆-Xaa₁₇-Cys-Xaa₁₈-Xaa₁₉-Xaa₂₀-Xaa₂₁-Xaa₂₂-Xaa₂₃-Xaa₂₄-Cys-Xaa₂₅-Xaa₂₆Xaa₂₇-Xaa₂₈-Xaa₂₉-Xaa₃₀ (SEQ ID NO:1),
wherein Xaa₁ is des-Xaa₁, Pro, hydroxy-Pro (Hyp), Arg, Lys, ornithine, homo-Lys, homoarginine, nor-Lys, N-methyl-Lys, N,N′-dimethyl-Lys, N,N′,N″-trimethyl-Lys or any synthetic basic amino acid; Xaa₂ is des-Xaa₂, Ala, Gly, Asp, Glu, γ-carboxy-glutamate (Gla), any synthetic acidic amino acid, Thr, Ser, g-Thr (where g is glycosylation), g-Ser, Trp (D or L), neo-Trp or halo-Trp (D or L) or Xaa₂ may be pyroglutamate if Xaa₁ is des-Xaa₁; Xaa₃ is Arg, Lys, ornithine, homo-Lys, homoarginine, nor-Lys, N-methyl-Lys, N,N′-dimethyl-Lys, N,N′,N″-trimethyl-Lys, any synthetic basic amino acid, Ser, Thr, g-Ser, g-Thr, Ala, an aliphatic amino acid bearing linear or branched saturated hydrocarbon chains such as Leu (D or L), Ile and Val or non-natural derivatives of the aliphatic amino acid, His, Glu, Gln, Gla, Asp, Asn or any synthetic acidic amino acid; Xaa₄ is Glu, Gla, Gln, Asp, Asn, any synthetic acidic amino acid, Lys, Arg, ornithine, homo-Lys, homoarginine, nor-Lys, N-methyl-Lys, N,N-dimethyl-Lys, N,N′,N″-trimethyl-Lys, any synthetic basic amino acid, Ala, an aliphatic amino acid bearing linear or branched saturated hydrocarbon chains such as Leu (D or L), Ile and Val or non-natural derivatives of the aliphatic amino acid, Ser, Thr, Pro, Hyp, g-Ser, g-Thr, g-Hyp or any synthetic hydroxylated amino acid; Xaa₅ is Lys, Arg, ornithine, homo-Lys, homoarginine, nor-Lys, N-methyl-Lys, N,N′-dimethyl-Lys, N,N′,N″-trimethyl-Lys, any synthetic basic amino acid, Tyr, meta-Tyr, ortho-Tyr, nor-Tyr, mono-halo-Tyr, di-halo-Tyr, O-sulpho-Tyr, O-phospho-Tyr, nitro-Tyr, an aliphatic amino acid bearing linear or branched saturated hydrocarbon chains such as Leu (D or L), Ile and Val or non-natural derivatives of the aliphatic amino acid, Glu, Gla, Gln, Asp, Asn, any synthetic acidic amino acid, Pro or Hyp; Xaa₆ is Trp (D or L), neo-Trp, halo-Trp (D or L), Gly, Tyr, meta-Tyr, ortho-Tyr, nor-Tyr, mono-halo-Tyr, di-halo-Tyr, O-sulpho-Tyr, O-phospho-Tyr, nitro-Tyr, Glu, Gla, Gln, Asp, Asn, any synthetic acidic amino acid; Xaa₇ is Glu, Gla, Gln, Asp, Asn, any synthetic acidic amino acid, Met, norleucine (Nle), Ala, an aliphatic amino acid bearing linear or branched saturated hydrocarbon chains such as Leu (D or L), Ile and Val or non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr, Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa₈ is Leu, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₉ is Pro, Hyp, Gly, an aliphatic amino acid bearing linear or branched saturated hydrocarbon chains such as Leu (D or L), Ile and Val or non-natural derivatives of the aliphatic amino acid; Xaa₁₀ is Thr, Ser, g-Thr, g-Ser, Ala, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Tip (D or L) or any synthetic aromatic amino acid; Xaa₁₁ is Pro, Hyp, Ser, Thr, g-Hyp, g-Ser, g-Thr or any hydroxylated amino acid; Xaa₁₂ is an aliphatic amino acid bearing linear or branched saturated hydrocarbon chains such as Leu i(D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa₁₃ is Pro, Hyp, an aliphatic amino acid bearing linear or branched saturated hydrocarbon chains such as Leu (D or L), Ile and Val or non-natural derivatives of the aliphatic amino acid, Lys, Arg, ornithine, homo-Lys, homoarginine, nor-Lys, N-methyl-Lys, N,N′-dimethyl-Lys, 14,N′,N″-trimethyl-Lys or any synthetic basic amino acid; Xaa₁₄ is Gly, His, Lys, Arg, ornithine, homo-Lys, homoarginine, nor-Lys, N-methyl-Lys, N,N′-dimethyl-Lys, N,N′,N″-trimethyl-Lys or any synthetic basic amino acid; Xaa₁₅ is des-Xaa₁₅, Ser, Thr, g-Ser, g-Thr, Val, Asn, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid;Xaa₁₆ is Met, Nle, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L), any synthetic aromatic amino acid, Arg, Lys, ornithine, homo-Lys, homoarginine, nor-Lys, N-methyl-Lys, N,N′-dimethyl-Lys, N,N′,N″-trimethyl-Lys or any synthetic basic amino acid; Xaa₁₇ is Pro, Hyp, Ser, Thr, g-Hyp, g-Ser, g-Thr, any hydroxylated amino acid, Ala, Met, Leu, Glu, Gla, Gln, Asp, Asn, any synthetic acidic amino acid, Tyr, meta-Tyr, ortho-Tyr, nor-Tyr, mono-halo-Tyr, di-halo-Tyr, O-sulpho-Tyr, O-phospho-Tyr, nitro-Tyr, His or Gly; Xaa₁₈ is Gly, Ala, Ser, Thr, Pro, Hyp, g-Ser, g-Thr, g-Hyp, Glu, Asn or Gln; Xaa₁₉ is Leu, Gly, Asn, Trp (D or L), neo-Trp or halo-Trp (D or L); Xaa₂₀ is des-Xaa₂₀, Leu or Trp (D or L), neo-Trp) or halo-Trp (D or L); Xaa₂₁ is des-Xaa₂₁ or an aliphatic amino acid bearing linear or branched saturated hydrocarbon chains such as Leu (D or L), Ile and Val or non-natural derivatives of the aliphatic amino acid; Xaa₂₂ is des-Xaa₂₂, Gly, Met, Nle, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₂₃ is des-Xaa₂₃, Pro, Hyp, Ala, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho)-Tyr, O-phospho-Tyr, nitro-Tyr, Trp (D or L), neo-Trp, halo-Trp (D or L) or any synthetic aromatic amino acid; Xaa₂₄ is Ala, an aliphatic amino acid bearing linear or branched saturated hydrocarbon chains such as Leu (D or L), Ile and Val or non-natural derivatives of the aliphatic amino acid, Phe, Tyr, meta-Tyr, ortho-Tyr, nor-Tyr, mono-halo-Tyr, di-halo-Tyr, O-sulpho-Tyr, O-phospho-Tyr, nitro-Tyr, Trp (D or L), neo-Trp, halo-Trp (D or L) or any synthetic aromatic amino acid; Xaa₂₅ is Ala, an aliphatic amino acid bearing linear or branched saturated hydrocarbon chains such as Leu (D or L), Ile and Val or non-natural derivatives of the aliphatic amino acid, Tyr, meta-Tyr, ortho-Tyr, nor-Tyr, mono-halo-Tyr, di-halo-Tyr, O-sulpho-Tyr, O-phospho-Tyr, nitro-Tyr or Phe; Xaa₁₆ is an aliphatic amino a(id bearing linear or branched saturated hydrocarbon chains such as Leu (D or L), Ile and Val or non-natural derivatives of the aliphatic amino acid; Xaa₂₇ is des-Xaa₁₇, Asp, Glu, Gla, Pro, Hyp, Ser, Thr, g-Hyp, g-Ser, g-Ser or any synthetic hydroxylated amino acid; Xaa₂₈ is des-Xaa₁₈, Glu, Gla, Gln, Asp, Asn, any synthetic acidic amino acid, Lys, Arg, ornithine, homo-Lys, homoarginine, nor-Lys, N-methyl-Lys, N,N′-dimethyl-Lys, N,N′,N″-trimethyl-Lys, any synthetic basic amino acid, Ile, Ser, Thr, g-Ser or g-Thr; Xaa₂₉ is des-Xaa₂₉, Pro, Hyp, Tyr, meta-Tyr, ortho-Tyr, nor-Tyr, mono-halo-Tyr, di-halo-Tyr, O-sulpho-Tyr, O-phospho-Tyr or nitro-Tyr; Xaa₃₀ is des-Xaa₃₀ or Phe, with the proviso that said μO-conopeptide is not MrVIA/B.

20. The substantially pure μO-conotopeptide of claim 19 selected from the group consisting of:
Ala-Cys-Arg-Gln-Xaa₁-Xaa₂-Xaa₃-Phe-Cys-Leu-Val-Xaa₄-Ile-Ile-Gly-Xaa₂-Ile-Xaa₂-Cys-Cys-Ala-Gly-Leu-Ile-Cys-Gly-Xaa₄-Phe-Val-Cys-Leu (SEQ ID NO:3);
Xaa₄-Thr-Cys-Leu-Xaa₁-Gln-Asp-Xaa₁-Phe-Cys-Ile-Ile-Xaa₄-Leu-Ile-Gly-Thr-Leu-Xaa₂-Cys-Cys-Ser-Gly-Leu-Ile-Cys-Gly-Pho-Phe-Val-Cys-Val-Xaa₄-Xaa₁-Xaa₄-Phe (SEQ ID NO:4);
Asp-Cys-Xaa₃-Ala-Asp-Gly-Ala-Phe-Cys-Gly-Ile-Xaa₄-Ile-Vla-Xaa₁-Asn-Xaa₅-Met-Cys-Cys-Ser-Asn-Leu-Cys-Ile-Phe-Ala-Cys-Val-Xaa₄-Xaa₃-Xaa₂ (SEQ ID NO:5);
Asp-Cys-His-Xaa₃-Arg-Xaa₅-Asp-Xaa₅-Cys-Xaa₄-Ala-Ser-Ile-Leu-Gly-Val-Ile-Xaa₂-Cys-Cys-Xaa₃-Gly-Leu-Ile-Cys-Phe-Ile-Ala-Phe-Cys-Ile (SEQ ID NO:6);
Asp-Cys-Gln-Xaa₃-Xaa₁-Xaa₅-Xaa₃-Phe-Cys-Ile-Val-Xaa₄-Ile-Leu-Gly-Phe-Val-Xaa₂-Cys-Cys-Xaa₄-Gly-Leu-Ile-Cys-Gly-Xaa₄-Phe-Val-Cys-Val-Asp-Ile (SEQ ID NO:7);
Xaa₄-Thr-Cys-Val-Ser-Xaa₂-Asn-Val-Phe-Cys-Gly-Val-Xaa₄-Leu-Val-Gly-Thr-Xaa₂-Leu-Cys-Cys-Ser-Gly-Leu-Val-Cys-Leu-Val-Val-Cys-Ile (SEQ ID NO:8);
Cys-Arg-Xaa₄-Arg-Gly-Met-Phe-Cys-Gly-Phe-Xaa₄-Xaa₁-Xaa₄-Gly-Xaa₄-Xaa₂-Cys-Cys-Asn-Gly-Xaa₅-Cys-Phe-Phe-Val-Cys-Ile (SEQ ID NO:9);
Arg-Xaa₅-Cys-Ala-Leu-Asp-Gly-Xaa₃-Leu-Cys-Ile-lle-Xaa₄-Val-Ile-Gly-Ser-Ile-Phe-Cys-Cys-His-Gly-Ile-Cys-Met-Ile-Xaa₂-Cys-Val (SEQ ID NO:10);
Asp-Cys-Arg-Xaa₄-Val-Gly-Gln-Xaa₂-Cys-Gly-Ile-Xaa₄-Xaa₂-Xaa₁-His-Asn-Xaa₅-Arg-Cys-Cys-Ser-Gln-Leu-Cys-Ala-Ile-Ile-Cys-Val-Ser (SEQ ID NO:1);
Gly-Cys-Leu-Asp-Xaa₄-Gly-Xaa₂-Phe-Cys-Gly-Thr-Xaa₄-Phe-Leu-Gly-Ala-Xaa₂-Cys-Cys-Gly-Gly-Ile-Cys-Leu-Ile-Val-Cys-Ile-Xaa₃-Thr (SEQ ID NO:12);
Asp-Cys-Xaa₃-Ala-Asp-Gly-Ala-Phe-Cys-Gly-Ile-Xaa₄-Thr-Val-Xaa₁-Asn-Xaa₅-Met-Cys-Cys-Ser-Asn-Leu-Cys-Ile-Phe-Ala-Cys-Val-Xaa₄-Xaa₃-Xaa₂ (SEQ ID NO:14);
Asp-Cys-Gln-Xaa₃-Xaa₃-Xaa₅-Xaa₃-Phe-Cys-Ile-Val-Xaa₄-Ile-Leu-Gly-Phe-Val-Xaa₂-Cys-Cys-Xaa₄-Gly-Leu-Ile-Cys-Gly-Xaa₄-Phe-Val-Cys-Val (SEQ ID NO:15);
Ala-Cys-Arg-Gln-Xaa₁-Xaa₂-Xaa₃-Phe-Cys-Leu-Val-Xaa₄-Ile-Ile-Gly-Xaa₂-Ile-Xaa₂-Cys-Cys-Ala-Gly-Leu-Ile-Cys-Gly-Xaa₄-Phe-Val-Cys-Leu (SEQ ID NO:16); and
Xaa₄-Thr-Cys-Leu-Xaa₁-Gln-Asp-Xaa₁-Phe-Cys-Ile-Ile-Xaa₄-Leu-Ile-Gly-Thr-Leu-Xaa₂-Cys-Cys-Ser-Gly-Leu-Ile-Cys-Gly-Phe-Phe-Val-Cys-Val-Xaa₄-Xaa₁-Xaa₄-Phe (SEQ ID NO:17),
wherein Xaa₁ is Lys, N-methy-Lys, N,N-dimethyl-Lys or N,N,N-trimethyl-Lys; Xaa₂ is Tyr, mono-halo-Tyr, di-halo-Tyr, O-sulpho-Tyr, O-phospho-Tyr or nitro-Tyr; Xaa₃ is Glu or gamma-carboxy-Glu (Gla); Xaa₄ is Pro or hydroxy-Pro; Xaa₅ is Trp or halo-Trp; and the C-terminus contains a carboxyl or amide group.

21. A pharmaceutical composition comprising the μO-conopeptide of claim 19 or a pharmaceutically acceptable salt or solvate thereof and a pharmaceutically acceptable carrier.

22. A pharmaceutical composition comprising the μO-conopeptide of claim 20 or a pharmaceutically acceptable salt or solvate thereof and a pharmaceutically acceptable carrier.
